(19) United States
(12) Patent Application Publication
(10) Pub. No.: US 2020/0362381 A1
(43) Pub. Date: Nov. 19, 2020

(54) MICROBIAL PRODUCTION OF NICOTINAMIDE RIBOSIDE

(71) Applicant: DSM IP ASSETS B.V., TH HEERLEN (NL)

(72) Inventors: Adam LAWRENCE, Kaiseraugst (CH); Celine VIAROUGE, Kaiseraugst (CH)

(21) Appl. No.: 16/614,680

(22) PCT Filed: May 17, 2018
(86) PCT No.: PCT/EP2018/063020

§ 371 Date: Nov. 18, 2019

Related U.S. Application Data
(60) Provisional application No. 62/508,309, filed on May 18, 2017.
(60) Provisional application No. 62/525,139, filed on Jun. 26, 2017.

(51) Int. Cl. C12P 19/30; C12N 9/00

(52) U.S. Cl. CPC C12P 19/30; C12R 1/01; C12N 9/93

(57) ABSTRACT

The present invention is directed to microbial production of nicotinamide mononucleotide using a genetically modified fungus.

This application is the U.S. national phase of International Application No. PCT/EP2018/063020 filed May 17, 2018 which designated the U.S. and claims priority to U.S. Provisional Patent Application Nos. 62/508,309 filed May 18, 2017, and 62/525,139 filed Jun. 26, 2017, the entire contents of each of which are hereby incorporated by reference.

### REFERENCE TO SEQUENCE LISTING SUBMITTED ELECTRONICALLY

The content of the electronically submitted sequence listing (Name: 4662_3895_ST25.txt; Size: 204 kilobytes; and Date of Creation: Mar. 6, 2020) is incorporated herein by reference in its entirety.

The present invention is directed to microbial production nicotinamide mononucleotide using a genetically modified bacterium.

Nicotinamide mononucleotide (NMN) is a pyridine-nucleoside form of vitamin B3 that functions as a precursor to nicotinamide adenine dinucleotide (NAD+). It is believed that high dose nicotinic acid can help to elevate high-density lipoprotein cholesterol, lowers low-density lipoprotein cholesterol and lower free fatty acids, although its mechanism has not been completely understood. NMN has been synthesized chemically in the past. The biological pathways leading to the synthesis of NMN are known but producing NMN at an industrial level remains a challenge.

Thus, it is desirable to identify new methods for producing NMN more efficiently, in particular for biotechnological production of NMN.

Surprisingly, the inventors have now found a novel method for significantly increasing the production rate of nicotinamide mononucleotide and created expression vectors and host cells useful in such methods.

In particular, the present invention is directed to a genetically modified bacterial strain capable of converting nicotinic acid mononucleotide (NaMN) to nicotinamide mononucleotide (NMN), wherein said strain comprising nicotinic acid mononucleotide amidating protein (NadE*) activity and reducing of nicotinamide mononucleotide nucleosidase activity, wherein the bacterium with said at least one modification produces an increased amount of NMN than the bacterium without any of said modifications.

The genetically modified bacterial strain according to the present invention may further comprise one or more additional modifications including one or more modification(s) being selected from the group consisting of:

(a) increasing L-aspartate oxidase activity;
(b) increasing quinolate synthase activity;
(c) increasing quinolate phoshoribosyltransferase activity;
(d) reducing the activity of a protein which functions to repress NAD+ biosynthesis by repressing transcription of nadA, nadB, nadC genes or combinations thereof;
(e) reducing NMN transporter protein activity;
(f) reducing nicotinic acid mononucleotide adenyltransferase activity;
(g) reducing nicotinamide mononucleotide amidohydrolase activity; and
(h) reducing purine nucleoside phosphorylase activity.

Thus, a genetically modified bacterial strain according to the present invention comprises a polynucleotide sequence encoding a polypeptide having NadE* activity, said polynucleotide being in particular of bacterial origin. Thus, the bacterial strain is modified via introduction of a heterologous gene, in particular a bacterial origin, wherein the heterologous gene encodes a polypeptide having NadE* activity and reducing of nicotinamide mononucleotide nucleosidase activity, wherein the bacterium with said at least one modification produces an increased amount of NMN than the bacterium without any of said modifications.

Preferably, the gene encoding a polypeptide having nicotinamide mononucleotide nucleosidase activity to be reduced comprises an amino acid sequence according to SEQ ID NOs: 57, 58, or 59, or a variant of said polypeptide, wherein said polypeptide has a nucleosidase activity for converting nicotinamide mononucleotide to nicotinamide riboside.

The gene encoding polypeptide having NadE* activity which is used for the purpose of the present invention might be originated from any bacterial source, including but not limited to a microorganism selected from the group consisting of Francisella, Dichelobacter, Mannheimia, and Actinobacillus, such as e.g. Francisella tularensis, Francisella sp. FSC1006, Francisella guangzhouensis, Francisella sp. TX077308, Francisella philomiragia, Francisella noatunensis, Francisella persica, Francisella cf. novicida 3523, Francisella tularensis, Dichelobacter nodosus, Mannheimia succinoproducens, or Actinobacillus succinogenes, in particular selected from the group consisting of F. tularensis, F. sp. FSC1006, F. guangzhouensis, F. sp. TX077308, F. philomiragia subsp. philomiragia ATCC 25017, F. philomiragia strain O #319-036 [FSC 153], F. noatunensis supbsp. orientalis str. Toba 04, F. philomiragia strain GA01-2794, F. persica ATCC VR-331, F. cf. novicida 3523, F. tularensis subsp. novicida D9876, F. tularensis subsp. novicida F6168, F. tularensis subsp. tularensis strain NIH B-38, F. tularensis subsp. holarctica F92, Dichelobacter nodosus VCS1703A, Mannheimia succinoproducens MBEL55E, and Actinobacillus succinogenes.

Preferably, the polypeptide having NadE* activity comprises an amino acid sequence with at least 50%, 60%, 70%, 80%, 90%, 95%, 97%, 99% or 100% identity to an amino acid sequence selected from a sequence according to SEQ ID NO: 1, 3, 4, 5, 6, 7, 8, 9, 10, 11, 12, 13, 14, 15, 16, 17 or 18, wherein said polypeptide has a nicotinic acid amidating activity for converting nicotinic acid mononucleotide to nicotinamide mononucleotide. Said polypeptide might be encoded by a polynucleotide including a sequence according to SEQ ID NO: 2, 19 to 40, and 43 to 47.

In particular, a polypeptide having NadE* activity is selected from one of the following sequences according to Table 1.

TABLE 1
list of NadE* amino acids sequences

| SEQ ID No. | | |
|---|---|---|
| [aa/nt] | origin | Accession no. |
| 1 | Francisella tularensis FtNadE* | YP_170217 |
| 3/24 | Francisella sp. FSC1006 | WP_040008427.1 |
| 4/25 | Francisella guangzhouensis | WP_039124332.1 |
| | 08HL01032 | |
| 5/23 | Francisella sp. TX077308 | WP_013922810.1 |
| 6 | Francisella philomiragia subsp. | WP_004287429.1 |
| | philomiragia ATCC 25017 | |
| 7 | Francisella philomiragia strain | WP_042517896.1 |
| | O#319-036 [FSC 153] | |
| 8 | Francisella noatunensis supbsp. | WP_014714556.1 |
| | orientalis str. Toba 04 | |
| 9 | Francisella philomiragia strain | WP_044526539.1 |
| | GA01-2794 | |
| 10/26 | Francisella persica ATCC VR-331 | WP_064461307.1 |
| 11/22 | Francisella cf. novicida 3523 | WP_014548640.1 |
| 12 | Francisella tularensis subsp. | WP_003037081.1 |
| | novicida D9876 | |
| 13 | Francisella tularensis subsp. | WP_003034444.1 |
| | novicida F6168 | |
| 14 | Francisella tularensis subsp. | WP_003025712.1 |
| | tularensis strain NIH B-38 | |
| 15 | Francisella tularensis subsp. | WP_010032811.1 |
| | holarctica F92 | |
| 16/20 | Dichelobacter nodosus VCS1703A | WP_011927945.1 |
| 17/19 | Mannheimia succinoproducens | WP_011201048.1 |
| | MBEL55E | |
| 18/21 | Actinobacillus succinogenes | WP_012072393.1 |

According to an embodiment of the present invention, such modified strain as described herein which is able to convert NaMN to NMN, is used in a process for producing NMN. In particular, a process according to the present invention is comprising culturing said strain under conditions effective to produce NMN and recovering NMN from the medium.

Thus, the present invention is related to a process for production of NMN, comprising:

(a) culturing a genetically modified bacterial strain capable of the conversion of NaMN to NMN as described herein under conditions effective to produce NMN,
(b) recovering NMN from the medium,
wherein the bacterial strain is encoding a heterologous polypeptide having NadE* activity and reducing of nicotinamide mononucleotide nucleosidase activity, wherein the bacterium with said at least one modification produces an increased amount of NMN than the bacterium without any of said modifications.

The present invention is also directed to a genetically modified bacterium characterized by that as a result of the genetic modification, the bacterium produces NMN and can accumulate the produced NMN to at least 100 mg/L in the fermentation broth in which the bacterium is grown.

In one embodiment, the genetically modified bacterial strain as described herein furthermore comprises increased L-aspartate oxidase (NadB) activity, which might be achieved by either increasing the activity of the endogenous gene or by introducing a heterologous gene from bacteria, including a gene comprising a nucleic acid sequence encoding a polypeptide according to SEQ ID NO: 41, 42, 79 or 80 or a variant of said polypeptide.

In another embodiment, the genetically modified bacterial strain as described herein furthermore comprises increased quinolinate synthase (NadA subunit A) activity, which might be achieved by either increasing the activity of the endogenous gene or by introducing a heterologous gene from bacteria, including a gene comprising a nucleic acid sequence encoding a polypeptide according to SEQ ID 76, 77 or 78, or a variant of said polypeptide. This modification might be combined with further modification, e.g. increased L-aspartate oxidase activity as described herein.

In another embodiment, the genetically modified bacterial strain as described herein furthermore comprises increased quinolinate phoshoribosyltransferase (NadC) activity, referably wherein the gene is originated from bacteria, which might be achieved by either increasing the activity of the endogenous gene or by introducing a heterologous gene from bacteria, including a gene comprising a nucleic acid sequence encoding a polypeptide according to SEQ ID NOs: 81, 82 or 83, or a variant of said polypeptide. This modification might be combined with further modification, e.g. increase L-aspartate oxidase activity and/or increased quinolate synthase activity as described herein.

In a further embodiment, the genetically modified bacterial strain as described herein furthermore comprises a reduction in the activity of a protein which functions to repress NAD+ biosynthesis by repressing transcription of nadA, nadB, nadC genes or combinations thereof, such as e.g. mutation in the endogenous gene encoding a repressor of NadA, NadB and/or NadC activity, including a gene comprising a nucleic acid sequence encoding a polypeptide according to SEQ ID 51, 52, or 53, or a variant of said polypeptide. This modification might be combined with further modification, e.g. increased L-aspartate oxidase activity and/or increased quinolate synthase activity and/or increased quinolate phoshoribosyltransferase activity as described herein.

In another embodiment, the genetically modified bacterial strain as described herein furthermore comprises a reduction in NMN transporter protein activity, such as e.g. mutation in the endogenous gene encoding NMN transporter protein activity. This modification might be combined with further modification, e.g. increased L-aspartate oxidase activity and/or increased quinolate synthase activity and/or increased quinolate phoshoribosyltransferase activity and/or reduction of NadA, NadB and/or NadC activity as described herein.

In a further embodiment, the genetically modified bacterial strain as described herein furthermore comprises a reduction in nicotinic acid mononucleotide adenyltransferase activity, such as e.g. mutation in the endogenous gene encoding nicotinic acid mononucleotide adenyltransferase (NadD) activity, including a gene comprising a nucleic acid sequence encoding a polypeptide according to SEQ ID 63, 64, or 65, or a variant of said polypeptide. This modification might be combined with further modification, e.g. increased L-aspartate oxidase activity and/or increased quinolate synthase activity and/or increased quinolate phoshoribosyltransferase activity and/or reduction of NadA, NadB and/or NadC activity and/or reduction of NMN transporter protein activity as described herein.

In a further embodiment, the genetically modified bacterial strain as described herein furthermore comprises a reduction in nicotinamide mononucleotide amidohydrolase activity, such as e.g. mutation in the endogenous gene encoding nicotinamide mononucleotide amidohydrolase activity, including a gene comprising a nucleic acid sequence encoding a polypeptide according to SEQ ID 60, 61, or 62, or a variant of said polypeptide. This modification might be combined with further modification e.g. increased L-aspartate oxidase activity and/or increased quinolate synthase activity and/or increased quinolate phoshoribosyltransferase activity and/or reduction of NadA, NadB and/or NadC activity and/or reduction of NMN transporter protein activity and/or reduction in nicotinic acid mononucleotide adenyltransferase activity as described herein.

In a further embodiment, the genetically modified bacterial strain as described herein furthermore comprises a reduction in purine nucleoside phosphorylase activity, such as e.g. mutation in the endogenous gene encoding purine nucleoside phosphorylase activity, including a gene comprising a nucleic acid sequence encoding a polypeptide according to SEQ ID 71, 72, 73, 74 or a variant of said polypeptide. This modification might be combined with further modification, e.g. increased L-aspartate oxidase activity and/or increased quinolate synthase activity and/or increased quinolate phoshoribosyltransferase activity and/or reduction of NadA, NadB and/or NadC activity and/or reduction of NMN transporter protein activity and/or reduction in nicotinic acid mononucleotide adenyltransferase activity and/or reduction of in nicotinamide mononucleotide amidohydrolase activity as described herein.

According to the present invention, the introduction of a gene encoding a polypeptide having NadE* activity, said polynucleotide being in particular of bacterial origin, results in increased production of NMN using said bacterial strain. Preferably, the polypeptide having NadE* activity is chosen from the ones listed in Table 1.

In a further aspect, the present invention is directed to a genetically modified bacterial strain capable of converting nicotinic acid mononucleotide (NaMN) to nicotinamide mononucleotide (NMN), said strain comprising a polypeptide having NadE* activity, said polypeptide having at least 50%, 60%, 70%, 80%, 90%, 95%, 97%, 99% or 100% identity to an amino acid sequence selected from a sequence according to any one of SEQ ID NO: 1, 3, 4, 5, 6, 7, 8, 9, 10, 11, 12, 13, 14, 15, 16, 17 or 18 and comprising a tyrosine at position 27 and/or a glutamine at position 133, and/or a arginine at position 236 in SEQ ID NO: 1, based on the ClustalW method of alignment when compared to SEQ ID NOS: 1 and 3 to 18 using the default parameters of GAP PENALTY=10, GAP LENGTH PENALTY=0.1, and Gonnet 250 series of protein weight matrix.

A suitable bacterial strain or host cell to be genetically modified according to the present invention can be any gram-positive bacteria or gram-negative bacteria, such as including but not limited to the genera Bacillus, Corynebacterium, Escherichia, Acinetobacter, Lactobacillus, Mycobacterium, Pseudomonas, and Ralstonia. Preferred are Bacillus subtilis, Corynebacterium glutamicum, Escherichia coli, Acinetobacter baylyi, and Ralstonia eutropha. These embodiments are not limited to particular species but rather encompass all major phyla of bacteria.

In one particular embodiment, the genetically modified bacterial strain is E. coli expressing a polypeptide having NadE* activity, such as e.g. a polypeptide according to any one of SEQ ID NO: 1, 3, 4, 5, 6, 7, 8, 9, 10, 11, 12, 13, 14, 15, 16, 17 or 18, in particular encoded by the FtNadE* or gene or its functional homologs resulting in production of excess NMN in the absence of native nucleosidase activity. Excess NMN can be exported and accumulates externally.

As used herein, scientific and technical terms used herein will have the meanings that are commonly understood by one of ordinary skill in the art.

The term NadE* or “polypeptide having NadE* activity” is used interchangeably herein and indicates an enzyme capable of catalyzing the conversion of NaMN to NMN.

The term “quinolinate synthase” indicates an enzyme capable of converting iminosuccinic acid and dihydroxyacetone phosphate to quinolinate and phosphate. The quinolinate synthase used in this invention can be from various organisms, such as E. coli, B. subtilis, C. glutamicum, etc. Examples of quinolinate synthase proteins include polypeptides having amino acid sequence according to SEQ ID NOs:76, 77, or 78. Genes encoding the quinolinate synthesis activity are provided under, for example, accession nos. ACX40525 (E. coli), NP_390663 (B. subtilis), and CAF19774 (C. glutamicum). The quinolinate synthase as defined includes functional variants of the above mentioned quinolinate synthases.

The term “quinolinate phosphoribosyltransferase” is a polypeptide comprising an amino acid sequence of any one of SEQ ID NOs: 81, 82, or 83 or a variant of said polypeptide, wherein said polypeptide has an activity of converting quinolinate and phosphoribosylpyrophosphate to nicotinamide mononucleotide and carbon dioxide.

The term “L-aspartate oxidase” indicates an enzyme capable of converting aspartic acid to iminosuccinic acid in an FAD dependent reaction. The L-aspartate oxidase used in this invention can be from various organisms, such as E. coli, B. subtilis, C. glutamicum, etc. Examples of L-aspartate oxidase proteins include polypeptides having amino acid sequence SEQ ID NO: 41, 42, 79 or 80. Genes encoding the L-aspartate oxidase activity are provided under, for example, accession nos. ACX38768 (E. coli) and NP_390665 (B. subtilis). The L-aspartate oxidase as defined includes functional variants of the above-mentioned L-aspartate oxidases.

The term “nicotinamide riboside transporter protein” indicates an enzyme capable of catalyzing the transport of nicotinamide riboside for importing nicotinamide riboside from the periplasm to the cytoplasm. The enzyme in S. cerevisiae is known as NRT1. The nicotinamide riboside transporter protein described in this invention is a native polypeptide of the host organism such as S. cerevisiae, A. niger, Y. lipolytica, etc. Examples of nicotinamide riboside transporter proteins include polypeptides having amino acid sequences SEQ ID NOs: 54, 55, 56.

The term “nicotinamide mononucleotide phosphorylase” indicates an enzyme capable of catalyzing the phosphorolysis of nicotinamide mononucleotide to nicotinamide riboside. The enzyme in S. cerevisiae is known as PNP1. The nicotinamide mononucleotide phosphorylase protein described in this invention can also be a native polypeptide of the host organism such as S. cerevisiae, A. niger, Y. lipolytica, etc. Examples of nucleoside phosphorylase proteins include polypeptides having amino acid sequence SEQ ID NOs: 71, 72.

The term “nicotinamide mononucleotide amidohydrolase” indicates an enzyme capable of catalyzing the conversion of nicotinamide mononucleotide to nicotinic acid mononucleotide. The enzyme is commonly known as PncC. The nicotinamide mononucleotide amidohydrolase described in this invention can be from various organisms such as E. coli, B. subtilis, C. glutamicum, etc. The nucleoside hydrolase protein described in this invention can also be a native polypeptide of the host organism such as S. cerevisiae, A. niger, Y. lipolytica, etc. Examples of nicotinamide mononucleotide amidohydrolase proteins include polypeptides having amino acid sequences SEQ ID NOs: 60, 61, or 62.

The term “nicotinic acid mononucleotide adenyltransferase” indicates an enzyme capable of catalyzing the conversion of nicotinic acid mononucleotide to nicotinic acid adenine dinucleotide. The enzymes in S. cerevisiae is known as NMA1 and NMA2. The nicotinic acid mononucleotide adenyltransferase protein described in this invention is a native polypeptide of the host organism such as S. cerevisiae, A. niger, Y. lipolytica, etc. Examples of nicotinic acid mononucleotide adenyltransferase proteins include polypeptides having amino acid sequences SEQ ID NOs: 63, 64, 65.

The term “purine nucleoside phosphorylase” indicates an enzyme capable of catalyzing the conversion of nicotinamide riboside and phosphate to nicotinamide and ribose-1-phosphate. Common names for the enzyme are DeoD, PupG and Pdp. The purine nucleoside phosphorylase described in this in this invention is a native polypeptide of the host organism such as E. coli, B. subtilis, C. glutamicum, etc. Examples of purine nucleoside phosphorylase proteins include polypeptides having amino acid sequences SEQ ID NOs: 71, 72, 73, 74. Genes encoding the purine nucleoside phosphorylase activity are provided under, for example, accession nos. WP_003231176.1 (B. subtilis), WP_003243952.1 (B. subtilis), WP_0032300447.1 (B. subtilis), WP_000224877.1 (E. coli), and BAC00196.1 (C. glutamicum).

The relatedness between two amino acid sequences or between two nucleotide sequences is described by the parameter “sequence identity”. For purposes of the present disclosure, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

The term “nucleic acid construct” means a nucleic acid molecule, either single or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

The term “control sequences” means all components necessary for the expression of a polynucleotide encoding a polypeptide of the present disclosure. Each control sequence may be native or foreign to the polynucleotide encoding the polypeptide or native or foreign to each other. Such control sequences include, but are not limited to, a leader, polyadenylation sequence, peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

The term “operably linked” means a configuration in which a control sequence is placed at an appropriate position relative to the coding sequence of a polynucleotide such that the control sequence directs the expression of the coding sequence.

The term “expression” includes any step involved in the production of the polypeptide including, but not limited to, transcription, post-transcriptional modification, translation, post-translational modification, and secretion.

The term “expression vector” means a linear or circular DNA molecule that comprises a polynucleotide encoding a polypeptide and is operably linked to additional nucleotides that provide for its expression.

The term “host cell” means any bacterial cell type that is susceptible to transformation, transfection, transduction, and the like with a nucleic acid construct or expression vector comprising a polynucleotide encoding any one of the polypeptide sequences of the present disclosure. The term “host cell” encompasses any progeny of a parent cell that is not identical to the parent cell due to mutations that occur during replication.

The nadE gene product from E. coli, B. subtilis, and most characterized bacterial species, as well as all characterized eukaryotic species, utilizes nicotinic acid adenine dinucleotide as substrate for an amidation reaction to produce NAD+. By this native pathway, nicotinamide riboside (NR) is obtained by breakdown of nicotinamide adenine dinucleotide (NAD+), as in previously described work (U.S. Pat. No. 8,114,626 B2) or as shown in FIG. 2.

The host bacterial cell may be genetically modified by any manner known to be suitable for this purpose by the person skilled in the art. This includes the introduction of the genes of interest, such as the gene encoding the nicotinic acid amidating protein NadE*, into a plasmid or cosmid or other expression vector which are capable of reproducing within the host cell. Alternatively, the plasmid or cosmid DNA or part of the plasmid or cosmid DNA or a linear DNA sequence may integrate into the host genome, for example by homologous recombination or random integration. To carry out genetic modification, DNA can be introduced or transformed into cells by natural uptake or by well-known processes such as electroporation. Genetic modification can involve expression of a gene under control of an introduced promoter. The introduced DNA may encode a protein which could act as an enzyme or could regulate the expression of further genes.

Genetic modification of a microorganism can be accomplished using classical strain development and/or molecular genetic techniques. Such techniques known in the art and are generally disclosed for microorganisms, for example, in Sambrook et al., 1989, Molecular Cloning: A Laboratory Manual, Cold Spring Harbor Labs Press. The reference Sambrook et al., ibid., is incorporated by reference herein in its entirety.

Suitable vectors for construction of such an expression vector are well known in the art and may be arranged to comprise the polynucleotide operably linked to one or more expression control sequences, so as to be useful to express the required enzymes in a host cell, for example a bacterial cell as described above. For example, promoters including, but not limited to, T7 promoter, pLac promoter, nudC promoter, ushA promoter, pVeg promoter can be used in conjunction with endogenous genes and/or heterologous genes for modification of expression patterns of the targeted gene. Similarly, exemplary terminator sequences include, but are not limited to, the use of XPR1, XPR2, CPC1 terminator sequences.

As used herein, the term “specific activity” or “activity” with regards to polypeptides as described means its catalytic activity, i.e. its ability to catalyze formation of a product from a given substrate. The specific activity defines the amount of substrate consumed and/or product produced in a given time period and per defined amount of protein at a defined temperature. As used herein “reduction of activity” means to reduce the total quantity of an enzyme in a cell or to reduce the specific activity of said enzyme in order to effect a reduction in units of activity per unit biomass. As used herein “increased activity” means to increase the total quantity of an enzyme in a cell or to increase the specific activity of said enzyme in order to effect an increase in units of activity per unit biomass.

The genetically modified bacteria of the present disclosure also encompass bacteria comprising variants of the polypeptides as defined herein. As used herein, “functional variant” means that the variant sequence has similar or identical functional enzyme activity characteristics to the enzyme having the native amino acid sequence specified herein. With regards to polypeptides, it means a polypeptide in which the amino acid sequence differs from the base sequence from which it is derived in that a substitution, insertion, and/or deletion of one or more (several) amino acid residues at one or more (several) positions are made. A substitution means a replacement of an amino acid occupying a position with a different amino acid; a deletion means removal of an amino acid occupying a position; and an insertion means adding 1-3 amino acids adjacent to an amino acid occupying a position. For example, a functional variant of SEQ ID NOs: 1 and 3 to 18 has similar or identical nicotinic acid amidating protein FtNadE* activity characteristics as SEQ ID NOs:1 and 3 to 18, respectively. An example may be that the rate of conversion by a functional variant of SEQ ID NOs: 1 and 3 to 18, of nicotinic acid mononucleotide to nicotinamide mononucleotide, may be the same or similar, although said functional variant may also provide other benefits. For example, at least about 80%, 90%, 95%, 96%, 97%, 98%, 99% or 100% the rate will be achieved when using the enzyme that is a functional variant of SEQ ID NOs: 1 and 3 to 18, respectively.

A functional variant or fragment of any of the above SEQ ID NO amino acid sequences, therefore, is any amino acid sequence which remains within the same enzyme category (i.e., has the same EC number). Methods of determining whether an enzyme falls within a particular category are well known to the skilled person, who can determine the enzyme category without use of inventive skill. Suitable methods may, for example, be obtained from the International Union of Biochemistry and Molecular Biology.

Amino acid substitutions may be regarded as “conservative” where an amino acid is replaced with a different amino acid with broadly similar properties. Non-conservative substitutions are where amino acids are replaced with amino acids of a different type. By “conservative substitution” is meant the substitution of an amino acid by another amino acid of the same class, in which the classes are defined as follows:

### Class Amino Acid Examples:

### Nonpolar: A, V, L, I, P, M, F, W

Uncharged polar: G, S, T, C, Y, N, Q

### Acidic: D, E

### Basic: K, R, H.

Nicotinamide mononucleotide compounds produced according to the present disclosure can be utilized in any of a variety of applications, for example, exploiting their biological or therapeutic properties (e.g., controlling low-density lipoprotein cholesterol, increasing high-density lipoprotein cholesterol, etc.). For example, according to the present disclosure, nicotinamide ribose may be used in pharmaceuticals, foodstuffs, and dietary supplements, etc.

The nicotinamide mononucleotide produced by the method disclosed in this invention could have therapeutic value in improving plasma lipid profiles, preventing stroke, providing neuroprotection with chemotherapy treatment, treating fungal infections, preventing or reducing neurodegeneration, or in prolonging health and well-being. Thus, the present invention is further directed to the nicotinamide riboside compounds obtained from the genetically modified bacterial cell described above, for treating a disease or condition associated with the nicotinamide riboside kinase pathway of NAD+ biosynthesis by administering an effective amount of a nicotinamide riboside composition. Diseases or conditions which typically have altered levels of NAD+ or NAD+ precursors or could benefit from increased NAD+ biosynthesis by treatment with nicotinamide riboside include, but are not limited to, lipid disorders (e.g., dyslipidemia, hypercholesterolaemia or hyperlipidemia), stroke, neurodegenerative diseases (e.g., Alzheimer's, Parkinsons and Multiple Sclerosis), neurotoxicity as observed with chemotherapies, Candida glabrata infection, and the general health declines associated with aging. Such diseases and conditions can be prevented or treated by diet supplementation or providing a therapeutic treatment regime with a nicotinamide riboside composition.

It will be appreciated that, the nicotinamide mononucleotide compounds isolated from the genetically modified bacteria of this invention can be reformulated into a final product. In some other embodiments of the disclosure, nicotinamide riboside compounds produced by manipulated host cells as described herein are incorporated into a final product (e.g., food or feed supplement, pharmaceutical, etc.) in the context of the host cell. For example, host cells may be lyophilized, freeze dried, frozen or otherwise inactivated, and then whole cells may be incorporated into or used as the final product. The host cell may also be processed prior to incorporation in the product to increase bioavailability (e.g., via lysis).

In some embodiments of the disclosure, the produced nicotinamide riboside compounds are incorporated into a component of food or feed (e.g., a food supplement). Types of food products into which nicotinamide riboside compounds can be incorporated according to the present disclosure are not particularly limited, and include beverages such as milk, water, soft drinks, energy drinks, teas, and juices; confections such as jellies and biscuits; fat-containing foods and beverages such as dairy products; processed food products such as rice, bread, breakfast cereals, or the like. In some embodiments, the produced nicotinamide riboside compound is incorporated into a dietary supplement, such as, for example, a multivitamin.

### FIGURES

FIG. 1. Biochemical pathway for synthesizing quinolinate from aspartate and dihydroxyacetone phosphate in the presence of NadA and NadB enzymes.

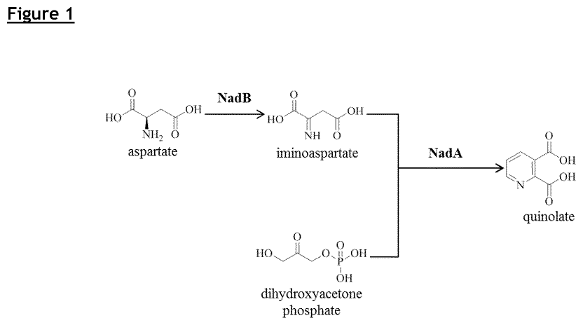

FIG. 2. Biochemical pathways and enzymes for synthesizing nicotinamide adenine dinucleotide.

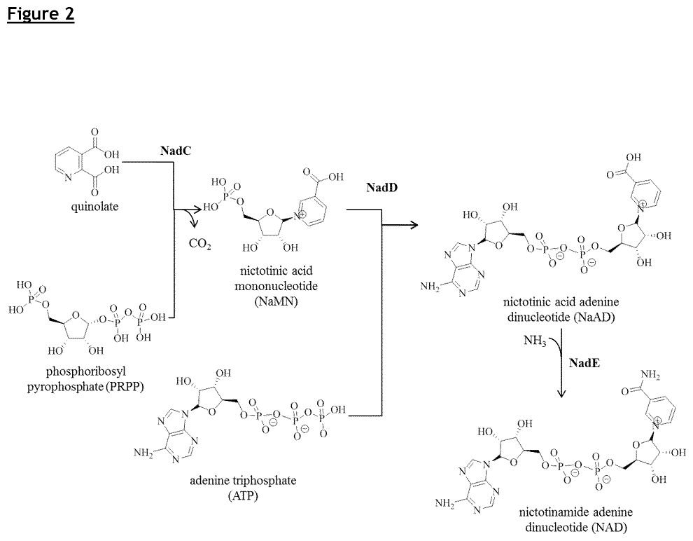

FIG. 3. Biochemical pathways useful for the production of nicotinamide riboside from NAD+ or intermediates of NAD+ biosynthesis.

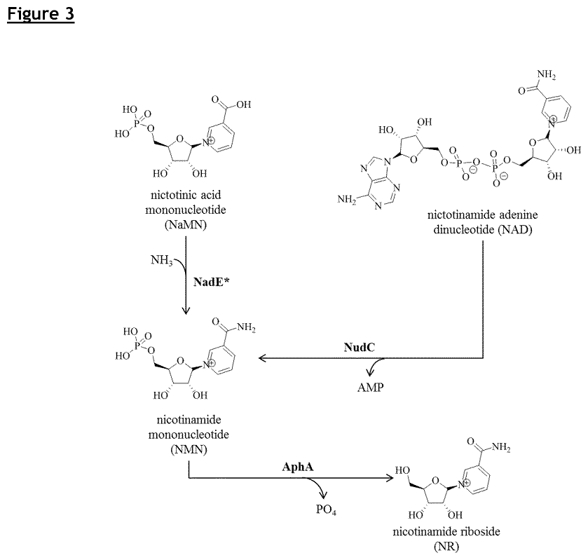

FIG. 4. Biochemical pathways with undesirable activities for nicotinamide riboside production.

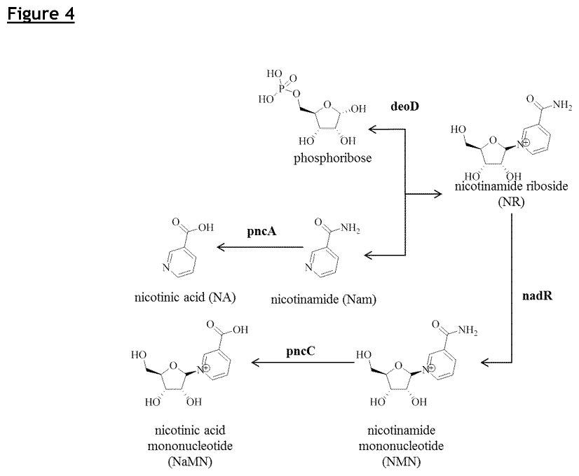

The following examples are intended to illustrate the invention without limiting its scope in any way.

### EXAMPLES

All basic molecular biology and DNA manipulation procedures described herein are generally performed according to Sambrook et al., Ausubel et al. or Barth. (J. Sambrook, E. F. Fritsch, T. Maniatis (eds). 1989. Molecular Cloning: A Laboratory Manual. Cold Spring Harbor Laboratory Press: New York; F. M. Ausubel, R. Brent, R. E. Kingston, D. D. Moore, J. G. Seidman, J. A. Smith, K. Struhl (eds.). 1998. Current Protocols in Molecular Biology. Wiley: New York); and G. Barth (ed.). 2013. Yarrowia lipolytica: Biotechnological Applications. Springer Science & Business Media, Berlin.

### Example 1: Identification of Sequences Coding for NaMN Amidating Activity (NadE*)

Sorci and co-workers identified the enzyme FtNadE* encoded by the genome of Francisella tularensis (SEQ ID NO:1) and demonstrated its ability to function both in vivo and in vitro as a nicotinamide mononucleotide (NaMN) amidating enzyme (Sorci L. e., 2009). In addition, they proposed that three amino acid residues were responsible for the enzyme's substrate preference for NaMN over NaAD: Y27; Q133; and R236. In order to identify additional sequences encoding this function, 50 unique nucleotide sequences derived from a BLAST search of the NCBI nr/nt database on 14 Sep. 2016 using default parameters for tBlastn with the amino acid sequence for FtNadE (SEQ ID NO:2) were translated and aligned using the Geneious alignment algorithm (Biomatters, LLLC.). 16 of these sequences had a conserved tyrosine, glutamine and arginine which aligned with Y27, Q133 and R236, respectively (i.e. contained a “Y-Q-R motif”) and were predicted to encode NaMN amidating enzymes (SEQ ID NOs: 3 to 18.

### Example 2: Genetic Constructs for Expression of NaMN Amidating Activity (NadE*) in E. coli

10 sequences encoding predicted nadE* open reading frames (SEQ ID NOs:2 and 19 to 27) were selected based on maximizing phylogenetic distance among the set of 16 predicted nadE* genes and were codon optimized for expression in E. coli using the Geneious codon optimization algorithm with the E. coli K-12 codon usage table and threshold to be rare set at 0.4. The optimized sequences (SEQ ID NOs: 28 to 37) were synthesized de novo by GenScript, Inc., and cloned into XhoI/NdeI digested pET24a(+) (Novagen, Inc.), also by GenScript, yielding the plasmids in Table 2. Plasmids were transformed into BL21(DE3), allowing for IPTG induction of the nadE* genes in order to induce NR synthesis and yielding the strains ME407, ME644, ME645, ME646, ME647, ME648, ME649, ME650, ME651, ME652 (Table 3).

TABLE 2
Plasmids used in this study

| plasmid | Description |
|---|---|
| pET24Ft | SEQ ID No. 37 (FtNadE*) cloned in pET24a(+) |
| pET24Dn | SEQ ID No. 29 (DnNadE*) cloned in pET24a(+) |
| pET24As | SEQ ID No. 30 (AsNadE*) cloned in pET24a(+) |
| pET24Fph | SEQ ID No. 31 (FphNadE*) cloned in pET24a(+) |
| pET24Fn | SEQ ID No. 32 (FnNadE*) cloned in pET24a(+) |
| pET24FspT | SEQ ID No. 33 (FspTNadE*) cloned in pET24a(+) |
| pET24FspF | SEQ ID No. 34 (FspFNadE*) cloned in pET24a(+) |
| pET24Fg | SEQ ID No. 35 (FgNadE*) cloned in pET24a(+) |
| pET24Fpe | SEQ ID No. 36 (FpeNadE*) cloned in pET24a(+) |
| pET24Mn | SEQ ID No. 28 (MnNadE*) cloned in pET24a(+) |
| pET24Ft-TGV | SEQ ID No. 42 (FtNadE*-TGV) cloned in |
| | pET24a(+) |
| pETMn-TGV | SEQ ID No. 43 (MnNadE*-TGV) cloned in |
| | pET24a(+) |
| pETFn-TGV | SEQ ID No. 44 (FnNadE*-TGV) cloned in |
| | pET24a(+) |
| pETFspT-TGV | SEQ ID No. 45 (FspTNadE*-TGV) cloned in |
| | pET24a(+) |
| pET-EcNadE | SEQ ID No. 46 (EcNadE) cloned in pET24a(+) |
| pBS-FnNadE | SEQ ID No. 38 cloned into pUC57 |
| pBS-FtNadE | SEQ ID No. 40 cloned into pUC57 |
| pBS-FspNadE | SEQ ID No. 41 cloned into pUC57 |
| pBS-MnNadE | SEQ ID No. 39 cloned into pUC57 |
| MB4124-FnNadE | SEQ ID No. 47 cloned into MB4124 |

TABLE 3
Strains used or described in this study

| Strain | Species | Genotype |
|---|---|---|
| BL21(DE3) | E. coli | fhuA2 [Ion] ompT gal (λ DE3) [dcm] |
| | | ΔhsdS λ DE3 = λ sBamHlo |
| | | ΔEcoRI-B int::(lacl:PlacUV5::T7 |
| | | gene1) i21 Δnin5 |
| ME407 | E. coli | BL21(DE3) pET24Ft |
| ME409 | E. coli | BL21(DE3) |
| ME644 | E. coli | BL21(DE3) pET24Dn |
| ME645 | E. coli | BL21(DE3) pET24As |
| ME646 | E. coli | BL21(DE3) pETFph |
| ME647 | E. coli | BL21(DE3) pET24 Fn |
| ME648 | E. coli | BL21(DE3) pET24FspT |
| ME649 | E. coli | BL21(DE3) pET24FspF |
| ME650 | E. coli | BL21(DE3) pET24Fg |
| ME651 | E. coli | BL21(DE3) pET24Fpe |
| ME652 | E. coli | BL21(DE3) pET24Mn |
| ME708 | E. coli | BL21(DE3) pETFn-rev |
| ME710 | E. coli | BL21(DE3) pETFspT-rev |
| ME712 | E. coli | BL21(DE3) pET24Ft-rev |
| ME714 | E. coli | BL21(DE3) pETMn-rev |
| ME683 | E. coli | BL21(DE3) pETEcNadE |
| BS168 | B. subtilis | Wildtype strain |
| BS6209 | B. subtilis | BS168 nadR::spec |
| ME479 | B. subtilis | BS168 deoD::tet |
| ME492 | B. subtilis | BS168 pupG::neo |
| ME496 | B. subtilis | BS168 nadR::spec deoD::tet |
| ME517 | B. subtilis | BS168 nadR::spec deoD::tet pupG::neo |
| ME795 | B. subtilis | ME517 amyE::cat pVeg-MsNadE* |
| ME805 | B. subtilis | ME517 amyE::cat pVeg-rbs4-FnNadE* |
| ME820 | B. subtilis | ME517 amyE::cat pVeg-FspNadE* |
| ME824 | B. subtilis | ME517 amyE::cat pVeg-rbs4-FtNadE* |
| ATCC13032 | C. glutamicum | Wildtype strain |
| ME763 | C. glutamicum | ATCC13032 MB4124-FnNadE |

### Example 3: Characterization of E. coli Strains Expressing NadE* Enzymes

To test the effect of NadE* expression on NR production, E. coli strains were inoculated from single colonies into LB medium and grown overnight (2 mL, 37° C., 15 mL test tube, 250 rpm, 50 μg/mL kanamycin). Precultures (200 μL) were used to inoculate 2 mL M9nC medium with or without 25 μM IPTG and grown in 24 well deep well plates (Whatman Uniplate, 10 mL, round bottom) sealed with an AirPore tape sheet (Qiagen) for three days (Infors Multitron Shaker, 800 rpm, 80% humidity). Samples were analyzed by LC-MS as described herein. Without plasmid, NR production was below the limit of quantification in the presence and absence of induction with 25 μM IPTG. Strains harboring plasmids for expression of NadE* enzymes produced up to 2.7 mg/L NR upon induction (Table 4).

TABLE 4
Nicotinamide riboside concentrations (mg/L) in E. coli shake plate
cultures upon IPTG induction (average of 2 cultures)

| Strain | Enzyme | No IPTG | 25 uM IPTG |
|---|---|---|---|
| ME407 | FtNadE* | <LOQ | 0.11 |
| ME409 | None | <LOQ | <LOQ |
| ME644 | DnNadE* | <LOQ | 0.28 |
| ME645 | AsNadE* | 0.08 | 0.31 |
| ME646 | FphNadE* | 0.02 | <LOQ |
| ME647 | FnNadE* | <LOQ | 1.91 |
| ME648 | FspTNadE* | 0.03 | 1.29 |
| ME649 | FspFNadE* | <LOQ | 0.82 |
| ME650 | FgNadE* | <LOQ | 0.64 |
| ME651 | FpeNadE* | 0.14 | 1.09 |
| ME652 | MnNadE* | 0.1 | 2.73 |

### Example 4: Increased NR Production in E. coli Requires a NadE* with Y27, Q133 and R236

To demonstrate the importance of the YQR motif for NR production, four of the 20 E. coli optimized NadE* sequences were altered to remove the residues which aligned to the Francisella tularensis Y27, Q133, R236 residues and replaced with the amino acid resides coded for in the Bacillus anthracis NadE (T, G, & V, respectively; SEQ ID NOs: 43 to 45). Site directed mutagenesis of the corresponding pET24a(+) plasmids was performed by GenScript, Inc, resulting in the plasmids in Table 2. Plasmids were transformed into BL21(DE3), allowing for IPTG induction of the nadE-TGV genes and yielding the strains, ME708, ME710, ME712, and ME714 (Table 3). These strains with a NadE-TGV failed to exhibit similar IPTG dependent increases in NR production to strains with NadE* (Table 5).

TABLE 5
Nicotinamide riboside concentrations (mg/L) in E. coli shake
plate cultures upon IPTG induction.

| | | No | |
|---|---|---|---|
| Strain | enzyme | IPTG | 50 uM IPTG |
| BL21(DE3) | none | <LOQ | <LOQ |
| BL21(DE3) | none | <LOQ | <LOQ |
| ME683 | EcNadE | <LOQ | <LOQ |
| ME683 | EcNadE | <LOQ | 0.01 |
| ME647 | FnNadE* | 0.04 | 0.92 |
| ME647 | FnNadE* | 0.02 | 0.75 |
| ME708 | FnNadE-TGV | <LOQ | 0.05 |
| ME708 | FnNadE-TGV | <LOQ | 0.07 |
| ME648 | FspTNadE* | <LOQ | 0.09 |
| ME648 | FspTNadE* | <LOQ | 0.16 |
| ME710 | FspTNadE-TGV | 0.01 | 0.01 |
| ME710 | FspTNadE-TGV | 0.02 | 0.01 |
| ME407 | FtNadE* | <LOQ | 0.1 |
| ME407 | FtNadE* | 0.02 | 0.12 |
| ME714 | FtNadE-TGV | 0.01 | <LOQ |
| ME714 | FtNadE-TGV | <LOQ | <LOQ |
| ME652 | MsNadE* | 0.07 | 1.11 |
| ME652 | MsNadE* | <LOQ | 0.96 |
| ME712 | MsNadE-TGV | <LOQ | 0.1 |
| ME712 | MsNadE-TGV | <LOQ | <LOQ |

### Example 5: Overexpression of E. coli NadE is not Sufficient to Observe Increased NR Production

To demonstrate that high levels of NaAD amidating activity (NadE) are insufficient to produce increased NR accumulation, the wildtype nadE open reading (SEQ ID NO: 46) frame was amplified via PCR from the genome of BL21(DE3) with primers M011159 and M011160 (Table 6) that added XhoI/NdeI restriction sites at the start and stop codons respectively. The PCR fragment was ligated into similarly digested pET24a(+), yielding plasmid pET24b+ nadEBL21.

This plasmid was transformed into BL21(DE3), allowing for IPTG induction of nadE and yielding the strain ME683. When tested for NR production alongside strains expressing NadE* sequences, this strain with additional expression of the E. coli NadE failed to exhibit IPTG dependent increases in NR concentration. (Table 5).

TABLE 6
Primers used in strain construction.

| | SEQ | | |
|---|---|---|---|
| Primer | ID NO: | Name | Use |
| 10444 | 84 | pDG1662_Pveg-I_pdxP | Amplification of all |
| | | Copy extraction (rev) | 3′ flanks |
| 10447 | 85 | amyE::nadEstar 5′ | Amplification of all |
| | | (reversed) (fwd) | 5′ flanks |
| 11222 | 86 | amyE-FnNadE 3′ for | |
| 11223 | 87 | amyE-FnNadE 3′ rev | |
| 11226 | 88 | amyE-FspNadE 3′ for | |
| 11227 | 89 | amyE-FspNadE 3′ rev | |
| 11230 | 90 | amyE-MsNadE 3′ for | |
| 11231 | 91 | amyE-MsNadE 3′ rev | Amplification of |
| | | | MsNadE gBlock |
| 11232 | 92 | PvegI MsNadE 5′ amyE for | Amplification of |
| | | | MsNadE gBlock |
| 11233 | 93 | PvegI MsNadE 5′ amyE rev | Amplification of |
| | | | MsNadE 5′ flank |
| 11234 | 94 | amyE-FtNadE 3′ for | |
| 11235 | 95 | amyE-FtNadE 3′ rev | |
| 11341 | 96 | rbs4 FnNadE rev | |
| 11342 | 97 | rbs4 FnNadE For | |
| 11351 | 98 | pVegI-FspNadE Rev | |
| 11352 | 99 | pVegI-FspNadE For | |
| 11353 | 100 | rbs4 FtNadE rev | |
| 11354 | 101 | rbs4 FtNadE For | |
| 11159 | 102 | XhoI-3′ NadE BL21 | Amplification of EcNadE |
| 11160 | 103 | NdeI 5′-NadE-BL21 | Amplification of EcNadE |

### Example 6: Construction B. subtilis Strains with Increased Basal Levels of NR Accumulation

In order to demonstrate efficacy of NadE* enzymes in promoting NR accumulation in a context of higher product accumulation, a host strain was engineered for increased basal levels of NR accumulation. E. coli strain DH5a, Corynebacterium glutamicum strain ATCC 13032 and B. subtilis strain 168 were grown overnight in rich media (LB for E. coli, BHI for C. glutamicum and B. subtilis) and inoculated 1:10 into 2 mL M9nC medium. After 24 hours, cultures were sampled for MS and relative NR levels were examined. B. subtilis NR production was higher than E. coli or C. glutamicum and was chosen as the host for further engineering.

Cassettes for the precise deletion of nadR, deoD, and pupG were constructed by long flanking PCR (LF-PCR). Flanking regions for each gene were obtained by amplification of BS168 genomic DNA (Roche High Pure PCR template preparation kit) with primers in Table 6, which were designed such that sequences homologous to the 5′ or 3′ region of the appropriate antibiotic resistance gene (spectinomycin, tetracycline, and neomycin, respectively, SEQ ID NOs: 48 to 50) were incorporated into the PCR product (Phusion Hot Start Flex DNA Polymerase, 200 nM each primer, initial denaturation 2 min @ 95° C., 30 cycles of: 30 sec @95° C.; 20 sec @ 50° C.; 60 sec @ 72° C., final hold 7 min at 72° C.). Antibiotic resistance genes were similarly amplified with primers to incorporate sequences homologous to the 5′ and 3′ flanking regions. PCR products were gel purified and used for LF-PCR with appropriate primers (Table 5) (Phusion Hot Start Flex DNA Polymerase, 200 nM each primer, 150 ng each PCR product, initial denaturation 30 sec @ 98° C., 35 cycles of: 30 sec @ 98° C.; 30 sec @ 55° C.; 360 sec @ 72° C.). LF-PCR product was purified and used for transformation of B. subtilis strains. BS168 was transformed with LF-PCR product via natural transformation (“Molecular Biological Methods for Bacillus”. 1990. Edited by C. R Harwood and S. M. Cutting. John Wiley and Sons) yielding BS6209 (nadR::spe), ME479 (deoD::tet), and ME492 (pupG::neo). Genomic DNA (prepared as above) from ME492 was used to transform BS6209, yielding ME496 (nadR::spe pupG::neo). Genomic DNA (prepared as above) from ME479 was used to transform ME496, yielding ME517 (nadR::spe pupG::neo deoD::tet).

### Example 7: Construction and Characterization of B. subtilis Strains Expressing NadE*

4 sequences encoding NadE* activity were codon optimized for expression in B. subtilis (Geneious codon optimization algorithm B. subtilis 168 codon usage table, threshold to be rare set at 0.4) and optimized sequences (SEQ ID NOs: 38-41) were synthesized as gBlocks by IDT. Cassettes for the expression of optimized NadE* sequences were generated by LF-PCR. A flanking region containing amyE 5′ region, cat (chloramphenicol resistance), pVegl promoter and a flanking region containing the amyE 3′ region, were amplified as above using appropriate primers (Table 6) and pDG1662 (Bacillus Genetic Stock Center) and gBlocks as templates. Gel purified flanking regions and gBlocks (above) were used for LF-PCR as above, and products were gel purified.

ME517 was transformed as above with the purified DNA and a transformant was colony purified, yielding strains ME795 (MsNadE*), ME805 (FnNadE*), ME820 (FspNadE*) and ME824 (FtNadE*). Strains were used to inoculate duplicate 1 mL cultures of BHI medium, and ME517 was inoculated in quadruplicate, in a 24 well shake plate and incubated at 37° C. overnight (as above). After 17 hours, plate was centrifuged, supernatant discarded and pellet was resuspended in 2 mL M9nC medium. Plates were placed back in incubator and grown a further 24 hours. NR was measured and strains harboring NadE* overexpression constructs produced on average between 72 and 133% more NR than the parent strain (Table 7).

### Example 8: Construction and Characterization of a Corynebacterium glutamicum Strain Expressing NadE*

In order to further demonstrate the general utility of these sequences for production of NR in bacteria, the sequence encoding FnNadE* was codon optimized for expression in C. glutamicum (Geneious codon optimization algorithm C. glutamicum codon usage table, threshold to be rare set at 0.4) and the optimized sequence (SEQ ID NO: 47) was synthesized as a gBlock by IDT with additional sequence upstream of the open reading frame encoding an EcoRI restriction site and Corynebacterium glutamicum consensus RBS and additional sequence downstream encoding a SmaI restriction site. The gBlock was digested with EcoRI/SmaI, yielding a 760 bp fragment which was ligated into similarly digested MB4124 yielding the plasmid MB4124-FnNadE*. MB4124 was derived from the cryptic C. glutamicum low-copy pBLI plasmid (see Santamaria et al. J. Gen. Microbiol, 130:2237-2246, 1984) by combining MB4094 (described in U.S. patent application 60/692,037) with an IPTG inducible promoter from pTrc99a (Gene. 1988 Sep. 30; 69(2):301-15.). C. glutamicum strain ATCC 13032 was transformed (Follettie, M. T., et al. J. Bacteriol. 167:695-702, 1993) with plasmid for IPTG inducible expression of FnNadE*S. cerevisiae strains engineered for the production of NR and/or NMN are inoculated in YPD medium and grown for 3 days at 30° C.

Single colonies were inoculated to 2 mL VY medium (+50 μg/mL kanamycin as appropriate) and grown at 30° C. overnight. 200 μL of this culture was used to inoculate 2 mL of AZ medium with 2% glucose (+10 μg/mL kanamycin where appropriate) and with varying levels of IPTG. NR was measured and strains harboring FnNadE* overexpression constructs displayed an IPTG dependent increase in NR production (Table 8).

### Example 9: Detection of Nicotinamide Riboside in Production Cultures

NR is analyzed by liquid chromatography/mass spectrometry (LCMS). After cultivation, 100 μL is diluted in 900 μL MS diluent (10% Water 10 mM Ammonium Acetate pH9.0, 90% acetonitrile) in 96 well deep well plates. Plates are centrifuged (10 min, 3000 rpm) and supernatant is transferred to a new plate for characterization. Supernatant is injected in 5 μl portions onto a HILIC UPLC column (Waters BEH Amide, 2.1×75 mm P/N 1860005657). Compounds are eluted at a flow rate of 400 μL min-1, after a 1-minute hold, using a linear gradient from 99.9% (10 mM ammonium acetate at pH 9.0 with 95% acetonitrile/5% Water) mobile phase D, to 70% (10 mM ammonium acetate pH 9.0 50/50 Acetonitrile/Water) mobile phase C, over 12 minutes, followed by a 1-minute hold in mobile phase C, and 5 minutes re-equilibration in mobile phase D (not shown). Eluting compounds are detected with a triple quadropole mass spectrometer using positive electrospray ionization. The instrument is operated in MRM mode and NR is detected using the transition m/z 123>80. NR is quantified by comparison to standard (Chromadex) injected under the identical condition. NMN is quantified by comparison to standard (Sigma Aldrich) injected under the identical condition.

### Example 10: Media Used for Bacterial Growth and Production Assays

1 liter of VY medium contains 25 g veal infusion broth (Difco), 5 g Bacto yeast extract (Difco)

1 liter of M9nC medium contains 50 g glucose, 6 g Na₂HPO₄, 3 g KH¬2¬PO₄, 0.5 g NaCl, 1 g NH₄Cl, 2 mM MgSO₄, 15 mg Na₂EDTA, 4.5 mg ZnSO₄*7 H₂O, 0.3 mg CoCl₂*6 H₂O, 1 mg MnCl₂*4 H₂O, 4.5 mg CaCl₂*2 H₂O, 0.4 mg Na₂MoO₄*2 H₂O, 1 mg H₃BO₃, and 0.1 mg KI.

1 liter of AZ medium contains 20 g glucose, 2 g NaCl, 3 g Na-Citrate, 0.1 g CaCl₂*2 H₂O, 4 g K₂HPO₄, 2 g KH₂PO₄, 7.5 g NH₄SO₄, 3.75 g urea, 0.5 g MgSO₄*7 H₂O, 450 μg thiamine, 450 μg biotin, 4 mg pantothenate, 15 mg Na2EDTA, 4.5 mg ZnSO₄*7 H₂O, 0.3 mg CoCl₂*6 H₂O, 1 mg MnCl₂*4 H₂O, 4.5 mg CaCl₂*2 H₂O, 0.4 mg Na₂MoO₄*2 H₂O, 1 mg H₃BO₃, and 0.1 mg KI.

### Example 11: Construction of B. subtilis Strains with Increased NMN Accumulation

In order to demonstrate NMN production in the absence of NMN nucleosidase activity, a strain engineered for increased levels of NR accumulation was converted to NMN production by removal of NMN nucleosidase activity.

A cassette for the precise deletion of yfkN in B. subtilis is constructed by long flanking PCR (LF-PCR). Flanking regions for each gene were obtained by amplification of BS168 genomic DNA (Roche High Pure PCR template preparation kit), with primers designed such that sequences homologous to the 5′ or 3′ region of the chloramphenicol resistance gene are incorporated into the PCR product (Phusion Hot Start Flex DNA Polymerase, 200 nM each primer, initial denaturation 2 min @ 95° C., 30 cycles of: 30 sec @ 95° C.; 20 sec @ 50° C.; 60 sec @ 72° C., final hold 7 min at 72° C.). The chloramphenicol resistance gene is similarly amplified with primers to incorporate sequences homologous to the 5′ and 3′ flanking regions. PCR products were gel purified and used for LF-PCR with appropriate primers (Table 5) (Phusion Hot Start Flex DNA Polymerase, 200 nM each primer, 150 ng each PCR product, initial denaturation 30 sec @ 98° C., 35 cycles of: 30 sec @ 98° C.; 30 sec @ 55° C.; 360 sec @ 72° C.). LF-PCR product was purified and used for transformation of B. subtilis strains.

ME805 is transformed with LF-PCR product via natural transformation (“Molecular Biological Methods for Bacillus”. 1990. Edited by C. R Harwood and S. M. Cutting. John Wiley and Sons). Isolated chloramphenicol resistant colonies are shown to produce increased NMN relative to the parental strain.

1. A genetically modified bacterial strain capable of converting nicotinic acid mononucleotide (NaMN) to nicotinamide mononucleotide (NMN), wherein said strain comprising nicotinic acid mononucleotide amidating protein (NadE*) activity and reducing of nicotinamide mononucleotide nucleosidase activity, wherein the bacterium with said at least one modification produces an increased amount of NMN than the bacterium without any of said modifications.

2. A genetically modified bacterial strain according to claim 1 which is selected from the group consisting of Bacillus, Corynebacterium, Escherichia, Acinetobacter, Lactobacillus, Mycobacterium, Pseudomonas, and Ralstonia, preferably selected from Bacillus subtilis, Corynebacterium glutamicum, Escherichia coli, Acinetobacter baylyi, and Ralstonia eutropha.

3. A genetically modified bacterial strain according to claim 1 expressing a heterologous polypeptide with nicotinic acid mononucleotide amidating protein (NadE*) activity, said polypeptide being selected from bacterial source, preferably from Francisella, Dichelobacter, Mannheimia, and Actinobacillus.

4. A genetically modified bacterial strain according to claim 1, wherein the polypeptide having NadE* activity comprises an amino acid sequence with at least 50%, 60%, 70%, 80%, 90%, 95%, 97%, 99% or 100% identity to an amino acid sequence selected from a sequence according to SEQ ID NO: 1, 3, 4, 5, 6, 7, 8, 9, 10, 11, 12, 13, 14, 15, 16, 17 or 18.

5. A genetically modified bacterial strain according to claim 1, further comprising one or more additional modifications including one or more modification(s) being selected from the group consisting of:
(a) increasing L-aspartate oxidase activity;
(b) increasing quinolate synthase activity;
(c) increasing quinolate phoshoribosyltransferase activity;
(d) reducing the activity of a protein which functions to repress NAD+ biosynthesis by repressing transcription of nadA, nadB, nadC genes or combinations thereof;
(e) reducing NMN transporter protein activity;
(f) reducing nicotinic acid mononucleotide adenyltransferase activity;
(g) reducing nicotinamide mononucleotide amidohydrolase activity; and
(h) reducing purine nucleoside phosphorylase activity.

6. A genetically modified bacterial strain according to claim 5, wherein L-aspartate activity is increased via overexpression of the endogenous gene or via expression of a heterologous gene encoding a polypeptide having L-aspartate activity.

7. A genetically modified bacterial strain according to claim 5, wherein quinolate synthase activity is increased via overexpression of the endogenous gene or via expression of a heterologous gene encoding a polypeptide having quinolate synthase activity.

8. A genetically modified bacterial strain according to claim 5, wherein quinolate phoshoribosyltransferase activity is increased via overexpression of the endogenous gene or via expression of a heterologous gene encoding a polypeptide having quinolate phoshoribosyltransferase activity.

9. A genetically modified bacterial strain according to claim 5, wherein the activity of a protein which functions to repress NAD+ biosynthesis by repressing transcription of nadA, nadB, nadC genes or combinations thereof is reduced.

10. A genetically modified bacterial strain according to claim 5, wherein the nicotinic acid mononucleotide adenyltransferase activity is reduced.

11. A genetically modified bacterial strain according to claim 5, wherein the nicotinamide mononucleotide amidohydrolase activity is reduced.

12. A genetically modified bacterial strain according to claim 5, wherein the purine nucleoside phosphorylase is reduced.

13. A process for production of NMN, comprising:
(a) culturing a genetically modified fungal strain according to claim 1 under conditions effective to produce NMN,
(b) recovering NMN from the medium,
wherein the fungal strain is encoding a heterologous polypeptide having NadE* activity.


SEQUENCE LISTING (103 sequences)

SEQ ID NO: 1 - protein, 249 aa, Francisella tularensis
  misc_feature (1)..(249): Francisella tularensis NadE amino acid sequence
MKIVKDFSPKEYSQKLVNWLSDSCMNYPAEGFVIGLSGGIDSAVAASLAVKTGLPTTALILPSDNNQHQDMQDALELIEMLNIEHYTISIQPAYEAFLASTQSFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 2 - DNA, 750 bp, Francisella tularensis
  misc_feature (1)..(750): Francisella tularensis NadE nucleotide sequence
atgaaaatagttaaagattttagtcctaaagaatattcacaaaagttagttaattggctaagtgatagttgtatgaattatcctgctgaaggatttgtgattggtcttagtggcggtatagattcagcagttgcggcttctttagctgtcaaaactggattaccaactacagctttaatactaccttcagataataatcaacaccaagatatgcaagatgctctagagcttattgaaatgcttaatattgaacattataccatttcgattcaaccagcttatgaggcttttcttgcttcaacgcaaagctttacaaatctacaaaacaatagacaacttgtgatcaagggaaatgctcaagcacgtttaaggatgatgtatttgtatgcctatgcgcaacaatataacagaatagttataggtactgataatgcttgtgagtggtatatgggatattttacaaaattcggtgatggggctgccgatatacttccactagttaatctcaaaaaatctcaagtttttgaattaggcaaatacctagatgtccctaaaaacatacttgataaagctccatctgcaggactatggcaaggacaaactgatgaggatgaaatgggtgtaacttatcaagaaattgatgatttcttagatggtaaacaagtttcagcaaaagctctagaaagaataaatttctggcataatcgtagtcaccataagagaaaattagctttaactcctaatttctag

SEQ ID NO: 3 - protein, 249 aa, Francisella sp.
  misc_feature (1)..(249): CP009574.1_translation Francisella sp. FSC1006, NadE
MSVVKNFKPNEYANKITEWLKDSCLNYPAEGFVVGISGGIDSAVAVSLAVNTGLPVTGLIMPSKNNDDKDTLDAIELAKKLNIEYHLIPIQPVYETFLDSAEDIKNSANDRQHVIKGNAQARFRMIYLYAYAQQNNRMVIGTDNACEWYMGYFTKFGDGAADILPLIKLKKSQVFELGSYLNVPNNILTKAPSAGLWLGQTDEAEMGVSYQEIDDFLDGKHVSDYALNQIKFWHNRSHHKRIMAKAPDF

SEQ ID NO: 4 - protein, 249 aa, Francisella guangzhouensis
  misc_feature (1)..(249): CP010427.1_translation Francisella guangzhouensis strain 08HL01032, NadE
MNVVKNFTPEKYSEKLIQWLTNSCIKYPAEGFVIGVSGGIDSAVCASLLSKTDLPTTAFILPSKNNSDQDMIDALELINKLNIPYHIIPIQPVYESFLKSTQLFTNPQNDRQNVIKGNAQARFRMMYLYAYAQQNNRIVVGTDNACEWYMGYFTKFGDGAADILPLINLKKSQVFELGKYLDVPRNILTKAPSAGLWQGQTDEGEMGVTYQEIDNFLDGKEVSPATFEKISYWHNRSHHKRKMALTPDF

SEQ ID NO: 5 - protein, 249 aa, Francisella sp.
  misc_feature (1)..(249): CP002872.1_translation Francisella sp. TX077308, NadE
MKIVKNFIVEQYSNNLIKWLKENCIKYPAEGFVIGISGGIDSAVAASLAVKTGLPTTALILPSKNNQDQDMRDGIELIENLNIEYHTVSIQPAYDTFIESTFNFTNSQNDRQHVIKGNAQARLRMMYLYAYAQQNNRIVIGTDNACEWYMGYFTKFGDGAADILPLINLKKSQVFELGKYLKVPKNIIQKDPSAGLWQGQTDEDEMGVTYKEIDDFLDGKEVSEKALERISFWHNRSHHKRSMAFTPNF

SEQ ID NO: 6 - protein, 249 aa, Francisella philomiragia
  misc_feature (1)..(249): CP000937.1_translation Francisella philomiragia subsp. philomiragia ATCC 25017, NadE
MKIIKNFIAEEYSKKLIEWLKKICINYPAEGFVIGISGGIDSAVAASLAVKTGLPTTALILPSKNNQDQDMKDGLELIKNLDIEHHIVPIQPAYDTFIESTLNFTNSQNDRQHVIKGNAQARLRMMYLYAYAQQNNRIVIGTDNACEWYMGYFTKFGDGAADILPLVNLKKSQVFEMGKYLKVPQNIIDKAPSAGLWQGQTDEDEMGVTYQEIDNFLDGKEVSAKALERINFWHNRSHHKRSMAFTPNF

SEQ ID NO: 7 - protein, 249 aa, Francisella philomiragia
  misc_feature (1)..(249): CP009442.1_translation Francisella philomiragia strain O#319-036 [FSC 153], NadE
MKIIKNFIAEEYSKKLIEWLKKICINYPAEGFVIGISGGIDSAVAASLAVKTGLPTTALILPSKNNQDQDMKDGLELIKNLDIEHHIVPIQPAYDTFIESTLNFTNSQNDRQHVIKGNAQARLRMMYLYAYAQQNNRIVIGTDNACEWYMGYFTKFGDGAADILPLVNLKKSQVFEMGEYLKVPQNIIDKAPSAGLWQGQTDEDEMGVTYQEIDNFLDGKEVSAKALERINFWHNRSHHKRSMAFTPNF

SEQ ID NO: 8 - protein, 249 aa, Francisella noatunensis
  misc_feature (1)..(249): CP003402.1_translation Francisella noatunensis subsp. orientalis str. Toba 04, NadE
MKIIKNFIAKEYSKKLIEWLKKICINYPAEGFVIGISGGIDSAVAASLAVKTGLPTTALILPSKNNQDQDMKDGLELIKNLDIEHHIVPIQPAYDTFIESTFNFTNAQNNRQHVIKGNAQARLRMMYLYAYAQQNNRIVIGTDNACEWYMGYFTKFGDGAADILPLVNLKKSQVFELGKYLKVPQNIIDKAPSAGLWQGQTDEDEMGVSYKEIDDFLDGKEVSEKALERINFWHNRSHHKRSIAFTPDF

SEQ ID NO: 9 - protein, 249 aa, Francisella philomiragia
  misc_feature (1)..(249): CP009440.1_translation Francisella philomiragia strain GA01-2794, NadE
MKIIKNFIVEKYSKKLIEWLKKICINYPAEGFVIGISGGIDSAVAASLAVKTGLPTTALILPSKNNQDQDMKDGLELIKNLDIEHHIVPIQPAYDTFIESTFNFTNAQNNRQHVIKGNAQARLRMMYLYAYAQQNNRIVIGTDNACEWYMGYFTKFGDGAADILPLVNLKKSQVFELGKYLKVPQNIIDKAPSAGLWQGQTDEDEMGVSYKEIDDFLDGKEVSEKALERINFWHNRSHHKRSIAFTPDF

SEQ ID NO: 10 - protein, 249 aa, Francisella persica
  misc_feature (1)..(249): CP013022.1_translation Francisella persica ATCC VR-331, NadE
MKIVKDFNIKEYSQKLIDWLSDTCMNYPAEGFVIGLSGGIDSAVAASLAVKTGLSTTALILPSKNNQHQDIQDALELADKINIEHHTITIQTVYETFLASIKKITNTERDRQLVIKGNAQARLRMMYLYAYAQQYNRVVIGTDNACEWYMGYFTKFGDGAADILPLVNLKKSHVFELGKYLGVPKNILDKAPSAGLWQGQTDEDEMGVTYQEIDDFLDGKQVSAKALERINFWHNRSHHKRKLALIPNF

SEQ ID NO: 11 - protein, 249 aa, Francisella cf.
  misc_feature (1)..(249): CP002558.1_translation Francisella cf. novicida 3523, NadE
MKIVKDFSPKEYSQNLVNWLSDTCINYPAEGFVIGISGGIDSAVAASLAVKTGLPTTALILPSKNNQHQDIQDALELVEKLNIEHHIVTIQPAYENFLASTQEFINTDNNRQLVIKGNAQARLRMMYLYAYAQQYNRIVIGTDNACEWYMGYFTKFGDGAADIFPLINLKKSQVFELGKYLDVPKNIIDKAPSAGLWQGQTDEDEMGVTYQEIDDFLDGKQISAKALERINFWHNRSHHKRKLALTPNF

SEQ ID NO: 12 - protein, 249 aa, Francisella tularensis
  misc_feature (1)..(249): CP009607.1_translation Francisella tularensis subsp. novicida D9876, NadE
MKIVKDFSPKEYSQKLVNWLSDSCMNYPAEGFVIGLSGGIDSAVAASLAVKTGLPTTALILPSDNNQHQDMQDALDLIEMLNIEHYTISIQPAYEAFLASTQRFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 13 - protein, 249 aa, Francisella tularensis
  misc_feature (1)..(249): CP009353.1_translation Francisella tularensis subsp. novicida F6168, NadE
MKIVKDFSPKEYSQKLVNWLSDSCMNYPAEGFVIGLSGGIDSAVAASLAVKTGLPITALILPSDNNQHQDMQDALDLIEMLNIEHYTISIQPAYEAFLASTQSFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 14 - protein, 249 aa, Francisella tularensis
  misc_feature (1)..(249): CP010115.1_translation Francisella tularensis subsp. tularensis strain NIH B-38, NadE
MKIVKDFSPKEYSQKLVNWLSDSCMNYPAEGFVIGLSGGIDSAVAASLAVKTGLPTTALILPSDNNQHQDMQDALELIEMLNIEHYTISIQLAYEAFLASTQSFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALEIINFWHNRSHHKRKLALTPNF

SEQ ID NO: 15 - protein, 249 aa, Francisella tularensis
  misc_feature (1)..(249): CP003932.1_translation Francisella tularensis subsp. holarctica F92, NadE
MKIVKDFSPKEYSQKLVNWLSDSCMNYPAEGFVIGLSGGIDSAVAASLAVKTGLPTTALILPSDNNQHQDMQDALELIEMLNIEHYTISIQPAYEAFLASTQSFTNLQNNRQLVIKGNAQTRLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 16 - protein, 239 aa, Dichelobacter nodosus
  misc_feature (1)..(239): CP000513.1_translation Dichelobacter nodosus VCS1703A, NadE
QYIDYLLVWLEEQRAHLYASDGYTLGVSGGIDSAVCLHLLAKTGKPVQALVLPINANANDCEDAELVLKNANISGNIIALDDVYTAAQNTLAPVLNRDYERMPVLNGNLMARLRMVMLYTVAQSHRSVVVGTDNAVEYYLGYFTKFGDGACDILPLAKLTKSEVGQLAKALGVPKKIREKAPSAGLWQGQTDENEIGVSYADLDAFLCGKTVDDAVREKIAYWHQRSHHKRMLPPMPEI

SEQ ID NO: 17 - protein, 245 aa, Unknown
  Mannheimia succiniciproducens
  misc_feature (1)..(245): AE016827.1_translation Mannheimia succiniciproducens MBEL55E, NadE
KRMKTAAYADYLIQWLENQRTELYGMDGYTLGVSGGIDSAVCAHLAARTGAPVQALILPAEVTSPSDVADAQATLESAGIDGQIISIAPWYDLIMQQLSPVLNSEPERVNVLKGNLMARLRMIALFTTAQSHRSIVLGTDNAAEWLTGYFTKFGDGAADVLPLAGLRKEQVFELGRYLGVPQSVLDKKPSAGLWAGQTDEAEMGVTYAEIDAYLRGETVSPQALQQIRFWHNRSHHKRMLPPKPK

SEQ ID NO: 18 - protein, 238 aa, Actinobacillus succinogenes
  misc_feature (1)..(238): CP000746.1_translation Actinobacillus succinogenes 130Z, NadE
YVDYLVRWLETQRTELYGMDGYTLGVSGGIDSAVCAHLAARTGAPVQALILPAEVTSPEDVADAQITLESAGIDGRIISIAPWYDLIMLQLTPALNAESERINVLKGNLMARLRMIALFTTAQSHRSIVLGTDNAAEMLTGYFTKFGDGAADVLPLARLRKEQVFELGRYLGVPKSVLEKKPSAGLWAGQTDEGEMGVSYAEIDAYLRGETVSPQALKQIQFWHNRSHHKRMLPPTPE

SEQ ID NO: 19 - DNA, 753 bp, Unknown
  Mannheimia succiniciproducens
  misc_feature (1)..(753): AE016827 Mannheimia succiniciproducens MBEL55E, NadE
atgaaaacggcagcatacgcagattatttaattcaatggctggaaaaccaacgaaccgaactttacgggatggacggctatacactgggcgtcagcggcggtattgacagcgccgtctgcgctcatttggcagcgcgcaccggcgcaccggtacaagccttaattttgcccgccgaagtaaccagtccgtcagatgtggcggatgcgcaagccacactggaaagcgccggtattgacggacaaataatttccattgcaccttggtacgatttaattatgcaacaactttccccggtattaaattccgaaccggagcgcgttaacgtattaaagggtaatttaatggcaagactgcgtatgattgcgctgtttaccacggcacaaagccatcgttctattgtgttaggcaccgataatgcggcggaatggctgacgggttattttaccaaattcggcgacggcgcagcggacgtactgcctttagcgggattgcgcaaagagcaggtatttgaactcggacgttatcttggcgtaccgcaaagcgtgctggataaaaaaccgagcgccggtttatgggcaggacaaacggacgaagctgaaatgggtgttacttatgcggaaatcgacgcttatctgcgcggcgaaaccgttagcccgcaggcattgcaacaaatccgtttctggcacaaccgttctcatcacaaacgtatgttgccacctaaaccgaaatcacccgatgaagcggagtgttaa

SEQ ID NO: 20 - DNA, 747 bp, Dichelobacter nodosus
  misc_feature (1)..(747): CP000513 Dichelobacter nodosus VCS1703A, NadE
atgaccgttcatcaatacatcgattatttactcgtgtggttagaagagcagcgcgctcatctttatgcatcagatggttatacgttgggcgtcagcggcggcatcgattccgccgtttgtctgcatttactcgccaaaacgggaaaacccgtgcaagcgttagttttgccgatcaatgcgaacgcgaacgattgtgaagatgccgaattagtgttaaaaaatgctaatatttccggcaatattatcgcgctcgatgatgtttataccgccgcacaaaacaccttggcgcctgttttaaatcgcgattatgaacgtatgcccgtattaaacggcaatttaatggcgcggctgcgtatggttatgctttataccgtggcgcaaagtcatcgttcggtggtcgtgggaacggataacgcggtggaatattatttaggttactttacaaaatttggcgacggcgcctgcgatattttgccgctggcaaaactgacaaaatcagaagtaggacaattggcaaaagcgttaggcgttccgaaaaaaatccgagaaaaagcgccgagcgcaggcttgtggcaagggcaaaccgatgaaaacgaaatcggcgtatcgtacgcggatttagatgcttttttgtgcggtaaaaccgttgatgatgccgtcagagaaaaaattgcttattggcatcaacgctcgcatcataaaagaatgttgccgccgatgccggaaatcggattatctttggcgtaa

SEQ ID NO: 21 - DNA, 750 bp, Actinobacillus succinogenes
  misc_feature (1)..(750): CP000746 Actinobacillus succinogenes 130Z, NadE
atgagaacggcagcatacgtagattatttagtgcgatggctggaaacccagcgtaccgaattatacggtatggacggctacacgctgggggtcagcggcggtatcgacagtgccgtttgcgcccatttagcggcacgcaccggcgcccccgtacaggcattgattttacccgccgaagtcaccagccctgaagatgtggcggatgctcagattaccttggaaagtgcaggtattgacgggcggattatttctatcgctccttggtacgatttaattatgctacaacttacccccgcattaaatgcggaatctgaacgcattaacgtattgaaaggtaacttaatggcgcgcttacgtatgatcgcattatttaccacggcgcaaagccaccgttctatcgtattgggtacggataacgccgccgaaatgttaacgggctatttcaccaaattcggcgacggtgcggcggacgtattgccgttagcgaggttgcgcaaagaacaggtattcgaattagggcgttatcttggcgtaccgaaatccgtgctggagaaaaaaccgagtgcgggcttatgggcggggcaaacggacgagggggaaatgggtgtcagttatgcggaaatcgacgcctatctgcgcggcgaaaccgtcagtccgcaggcgttaaagcagattcaattctggcacaaccgttctcatcacaaacgtatgctgccgccgacgccagaaccgccggatgaaatcgattaa

SEQ ID NO: 22 - DNA, 750 bp, Francisella cf.
  misc_feature (1)..(750): CP002558 Francisella cf. novicida 3523, NadE
atgaaaatagttaaagattttagtcctaaagaatattcacaaaatttagttaattggctaagtgatacttgtataaattatcctgctgaaggatttgtaatcggcattagcggtggtatagattcagctgttgcagcttctttagctgtcaaaactggattaccaactacagctttaatactaccttcaaaaaacaatcaacaccaagatatccaagatgctctagaacttgttgagaaacttaatattgaacatcatattgttacaattcaaccagcatacgaaaattttctagcatcaacacaggaatttataaatacagataataatagacaacttgtgatcaagggaaatgctcaagcacgtttaaggatgatgtatttatatgcctatgcccaacaatataacagaatagttataggtactgataatgcttgtgagtggtatatgggatattttacaaaatttggtgatggcgctgctgatatatttccgctaattaatcttaaaaaatcacaagtttttgaattaggtaaatacttagatgttccgaaaaatataattgataaagctccgtctgctggactatggcaaggacaaactgatgaggatgaaatgggcgtaacttatcaagaaattgatgatttcttagatggtaaacaaatttcagcaaaagccctagaaagaataaacttctggcataatcgtagtcatcataagagaaaactagctttaactcctaatttctaa

SEQ ID NO: 23 - DNA, 750 bp, Francisella sp.
  misc_feature (1)..(750): CP002872 Francisella sp. TX077308, NadE
atgaaaatagtaaaaaactttattgtagaacagtattctaataatttaataaaatggttaaaagagaattgcataaaatatcctgctgaaggttttgtgattggtattagtggtggtatcgattcggcagtagccgcatctttagcagtcaaaacaggattacctacaactgctctaatattgccatcgaagaacaatcaagaccaagatatgcgagatggaatagaactaatcgaaaatcttaatatagagtatcatactgtttcaatacaacctgcttatgacacgtttatagagtcaacatttaactttacaaactcacaaaatgatcgccaacatgttatcaaaggaaatgcccaagcgcgtcttagaatgatgtatttatatgcttatgctcagcaaaataatagaattgttataggtacagataacgcatgtgaatggtacatgggatatttcactaaatttggtgatggtgcagcagatatattaccacttattaatctcaaaaaatctcaagtttttgaactaggtaaatacttaaaagtgccaaaaaacattatccaaaaagatccttctgccggtctatggcaaggtcaaactgatgaggatgaaatgggtgtcacatacaaagaaattgatgacttcttagacggtaaagaagtctcagaaaaagctctcgaaagaataagcttctggcataatcgtagtcaccataaaagatccatggcttttacccctaatttttaa

SEQ ID NO: 24 - DNA, 750 bp, Francisella sp.
  misc_feature (1)..(750): CP009574 Francisella sp. FSC1006, NadE
atgagtgtagtaaaaaattttaaacctaatgaatatgccaataaaattactgaatggctgaaagactcttgtttaaattatcccgctgaaggttttgtggtaggtattagtggaggtatagattcagcagtagcagtctctttagcagtaaatactggactacctgttacagggctaataatgccatcaaaaaataatgatgataaagataccttagatgctatagaattagctaaaaaattaaatatagaatatcatctcatacccattcaaccagtatatgaaacatttctagattcagctgaagatatcaaaaacagtgctaatgaccgtcaacatgtaatcaaaggaaatgcacaagctcgttttagaatgatatacttgtatgcttacgctcagcaaaataatagaatggtaattggtacagataatgcttgtgaatggtatatgggctattttacaaaatttggagatggagccgctgatatactgcctcttataaaattaaaaaaatcacaagtttttgaattaggtagctatcttaatgtacccaataacatcctcacaaaagctccttccgcaggactttggcttggacaaactgatgaagcagagatgggggtttcatatcaagaaatagatgatttccttgatggtaaacatgtctcagattatgctcttaatcaaataaaattctggcataaccgtagtcatcataaaagaatcatggctaaggctccagatttttaa

SEQ ID NO: 25 - DNA, 753 bp, Francisella guangzhouensis
  misc_feature (1)..(753): Francisella guangzhouensis strain 08HL01032, NadE
atgaacgtagtaaaaaatttcactcctgaaaaatattcagaaaaacttatacaatggctcactaatagctgtataaaatatcctgcagaaggtttcgtaattggtgtaagtggtggtatagattctgcagtatgtgcatcacttttatccaaaactgatcttcctacaacagcttttatactaccatcaaaaaataactctgatcaagatatgatcgatgcattagaacttataaataaattaaatattccataccatataataccaatccagccagtttatgaaagttttctaaagtccacacagctatttacaaatccacaaaatgacagacaaaatgtcataaaaggtaacgctcaagctcgttttagaatgatgtatttatatgcttatgcacaacaaaataatcgtatagtagttggaacagataatgcttgtgaatggtatatgggttatttcaccaaatttggcgatggagctgctgatatactaccattaataaatcttaaaaagtcccaggtatttgagttaggtaaatacttagatgttccaaggaatatcctaactaaggcaccctctgctggtctttggcaaggccaaactgatgaaggtgaaatgggagttacttatcaggaaatagataattttctcgacggtaaagaagtatcgccagcaacttttgaaaaaataagctactggcataatcgctctcaccacaaaagaaagatggctttaacgccagattttaactaa

SEQ ID NO: 26 - DNA, 750 bp, Francisella persica
  misc_feature (1)..(750): CP013022 Francisella persica ATCC VR-331, NadE
atgaaaatagttaaagatttcaacatcaaagaatattcacaaaagttaattgattggctaagtgatacttgtatgaattaccctgctgaaggatttgtcattggtcttagcggtggtatagattcggcagttgcagcttctttagctgtcaaaactggattatcaactacagctttaatattaccatcaaaaaacaatcaacaccaagatatacaagatgctctagaacttgcagataaaattaatattgaacatcatactattacaattcaaacagtatacgaaacttttcttgcgtcaataaaaaaaattacaaataccgaacgtgatagacaacttgtcattaaaggaaatgctcaagctcgtttgaggatgatgtatttatatgcctatgctcaacaatataatagagtggttattggtactgataatgcttgtgaatggtatatgggatattttacaaagtttggtgatggtgctgctgatattcttccactagttaatctcaaaaaatctcacgtttttgaattaggtaaatacttaggtgttcctaaaaatatacttgataaagctccatctgctgggctatggcaaggacaaactgatgaagatgaaatgggcgtaacttatcaagaaattgatgatttcttagatggtaagcaagtttcagcgaaagctctagaaagaataaatttctggcataatcgtagtcatcataagagaaaactagctttaattcctaatttctaa

SEQ ID NO: 27 - DNA, 753 bp, Unknown
  Mannheimia succiniciproducens
  misc_feature (1)..(753): Optimized_AE016827 Mannheimia succiniciproducens MBEL55E, NadE
atgaaaacggcagcatacgcagattatctgattcaatggctggaaaaccaacgcaccgaactgtacggcatggacggctataccctgggcgtcagcggcggtattgacagcgccgtctgcgctcatctggcagcgcgcaccggcgcgccggtacaagccctgattctgccggcggaagtaaccagtccgtcagatgtggcggatgcgcaagccaccctggaaagcgccggtattgacggccaaattatttccattgcaccgtggtacgatctgattatgcaacaactgtccccggtactgaatagcgaaccggagcgcgttaacgtactgaaaggtaatctgatggcacgcctgcgtatgattgcgctgtttaccacggcacaaagccatcgttctattgtgctgggcaccgataatgcggcggaatggctgacgggttattttaccaaattcggcgacggcgcagcggacgtactgccgctggcgggcctgcgcaaagagcaggtatttgaactgggccgttatctgggcgtaccgcaaagcgtgctggataaaaaaccgagcgccggtctgtgggcaggccaaacggacgaagctgaaatgggtgttacctatgcggaaatcgacgcttatctgcgcggcgaaaccgttagcccgcaggcactgcaacaaatccgtttctggcacaaccgttctcatcacaaacgtatgctgccgccgaaaccgaaatcaccggatgaagcggagtgttaa

SEQ ID NO: 28 - DNA, 747 bp, Dichelobacter nodosus
  misc_feature (1)..(747): Optimized_CP000513 Dichelobacter nodosus VCS1703A, NadE
atgaccgttcatcaatacattgattatctgctggtgtggctggaagagcagcgcgctcatctgtatgcatcagatggttatacgctgggcgtcagcggcggcattgattccgccgtttgtctgcatctgctggccaaaacgggcaaaccagtgcaagcgctggttctgccgatcaatgcgaacgcgaacgattgtgaagatgccgaactggtgctgaaaaatgctaatatttccggcaatattatcgcgctggatgatgtttataccgccgcacaaaacaccctggcgccggttctgaatcgcgattatgaacgtatgccggtactgaacggcaatctgatggcgcgcctgcgtatggttatgctgtataccgtggcgcaaagtcatcgttcggtggtcgtgggcacggataacgcggtggaatattatctgggttactttaccaaatttggcgacggcgcctgcgatattctgccgctggcaaaactgaccaaatcagaagtaggccaactggcaaaagcgctgggcgttccgaaaaaaatccgcgaaaaagcgccgagcgcgggcctgtggcaaggccaaaccgatgaaaacgaaatcggcgtatcgtacgcggatctggatgcttttctgtgcggtaaaaccgttgatgatgccgtccgcgaaaaaattgcttattggcatcaacgctcgcatcataaacgtatgctgccgccgatgccggaaatcggcctgtctctggcgtaa

SEQ ID NO: 29 - DNA, 750 bp, Actinobacillus succinogenes
  misc_feature (1)..(750): Optimized_CP000746 Actinobacillus succinogenes 130Z, NadE
atgcgcacggcagcatacgtagattatctggtgcgctggctggaaacccagcgtaccgaactgtacggtatggacggctacacgctgggcgtcagcggcggtatcgacagtgccgtttgcgcccatctggcggcacgcaccggcgccccggtacaggcactgattctgccggcggaagtcaccagcccggaagatgtggcggatgctcagattaccctggaaagtgcaggtattgatggccgcattatttctatcgctccgtggtacgatctgattatgctgcaactgaccccggcactgaatgcggaatctgaacgcattaacgtactgaaaggtaacctgatggcgcgcctgcgtatgatcgcactgtttaccacggcgcaaagccaccgttctatcgtactgggtacggataacgccgccgaaatgctgacgggctatttcaccaaattcggcgacggtgcggcggacgtactgccgctggcgcgcctgcgcaaagaacaggtattcgaactgggccgttatctgggcgtaccgaaatccgtgctggagaaaaaaccgagtgcgggcctgtgggcgggccaaacggacgagggcgaaatgggtgtcagttatgcggaaatcgacgcctatctgcgcggcgaaaccgtcagtccgcaggcgctgaaacagattcaattctggcacaaccgttctcatcacaaacgtatgctgccgccgacgccggaaccgccggatgaaattgattaa

SEQ ID NO: 30 - DNA, 750 bp, Francisella cf.
  misc_feature (1)..(750): Optimized_CP002558 Francisella cf. novicida 3523, NadE
atgaaaattgttaaagattttagtccgaaagaatattcacaaaatctggttaattggctgagtgatacctgtattaattatccggctgaaggctttgtaatcggcattagcggtggtattgattcagcggttgcagcttctctggctgtcaaaaccggcctgccgaccaccgctctgattctgccgtcaaaaaacaatcaacaccaagatattcaagatgctctggaactggttgagaaactgaatattgaacatcatattgttaccattcaaccggcatacgaaaattttctggcatcaacccaggaatttattaataccgataataatcgccaactggtgatcaaaggcaatgctcaagcacgtctgcgcatgatgtatctgtatgcctatgcccaacaatataaccgcattgttattggtaccgataatgcttgtgagtggtatatgggctattttaccaaatttggtgatggcgctgctgatatttttccgctgattaatctgaaaaaatcacaagtttttgaactgggtaaatacctggatgttccgaaaaatattattgataaagctccgtctgctggcctgtggcaaggccaaaccgatgaggatgaaatgggcgtaacctatcaagaaattgatgatttcctggatggtaaacaaatttcagcaaaagccctggaacgcattaacttctggcataatcgtagtcatcataaacgcaaactggctctgaccccgaatttctaa

SEQ ID NO: 31 - DNA, 750 bp, Francisella sp.
  misc_feature (1)..(750): Optimized_CP002872 Francisella sp. TX077308, NadE
atgaaaattgtaaaaaactttattgtagaacagtattctaataatctgattaaatggctgaaagagaattgcattaaatatccggctgaaggttttgtgattggtattagtggtggtattgattcggcagtagccgcatctctggcagtcaaaaccggcctgccgaccaccgctctgattctgccgtcgaaaaacaatcaagaccaagatatgcgcgatggcattgaactgatcgaaaatctgaatattgagtatcataccgtttcaattcaaccggcttatgacacgtttattgagtcaacctttaactttaccaactcacaaaatgatcgccaacatgttatcaaaggcaatgcccaagcgcgtctgcgcatgatgtatctgtatgcttatgctcagcaaaataatcgcattgttattggtaccgataacgcatgtgaatggtacatgggctatttcaccaaatttggtgatggtgcagcagatattctgccgctgattaatctgaaaaaatctcaagtttttgaactgggtaaatacctgaaagtgccgaaaaacattatccaaaaagatccgtctgccggtctgtggcaaggtcaaaccgatgaggatgaaatgggtgtcacctacaaagaaattgatgacttcctggacggtaaagaagtctcagaaaaagctctggaacgcattagcttctggcataatcgtagtcaccataaacgcagcatggcttttaccccgaatttttaa

SEQ ID NO: 32 - DNA, 750 bp, Francisella sp.
  misc_feature (1)..(750): Optimized_CP009574 Francisella sp. FSC1006, NadE
atgagtgtagtaaaaaattttaaaccgaatgaatatgccaataaaattaccgaatggctgaaagactcttgtctgaattatccggctgaaggttttgtggtaggtattagtggcggtattgattcagcagtagcagtctctctggcagtaaataccggcctgccggttaccggcctgattatgccgtcaaaaaataatgatgataaagataccctggatgctattgaactggctaaaaaactgaatattgaatatcatctgattccgattcagccggtatatgaaacctttctggattcagcggaagatattaaaaacagtgctaatgaccgtcaacatgtaatcaaaggcaatgcacaagctcgttttcgcatgatttacctgtatgcttacgctcagcaaaataatcgcatggtaattggtaccgataatgcttgtgaatggtatatgggctattttaccaaatttggcgatggcgccgctgatattctgccgctgattaaactgaaaaaatcacaagtttttgaactgggtagctatctgaatgtaccgaataacatcctgaccaaagctccgagcgcgggcctgtggctgggccaaaccgatgaagcagagatgggcgtttcatatcaagaaattgatgatttcctggatggtaaacatgtctcagattatgctctgaatcaaattaaattctggcataaccgtagtcatcataaacgcatcatggctaaagctccggatttttaa

SEQ ID NO: 33 - DNA, 753 bp, Francisella guangzhouensis
  misc_feature (1)..(753): Optimized_CP010427 Francisella guangzhouensis strain 08HL01032, NadE
atgaacgtagtaaaaaatttcaccccggaaaaatattcagaaaaactgattcaatggctgaccaatagctgtattaaatatccggcagaaggtttcgtaattggtgtaagtggtggtattgattctgcggtatgtgcatcactgctgtccaaaaccgatctgccgaccaccgcttttattctgccgtcaaaaaataactctgatcaagatatgattgatgcactggaactgattaataaactgaatattccgtaccatattattccgatccagccggtttatgaaagttttctgaaatccacccaactgtttaccaatccgcaaaatgaccgccaaaatgtcattaaaggtaacgctcaagctcgttttcgcatgatgtatctgtatgcttatgcacaacaaaataatcgtattgtagttggcaccgataatgcttgtgaatggtatatgggttatttcaccaaatttggcgatggcgctgctgatattctgccgctgattaatctgaaaaaatcccaggtatttgagctgggtaaatacctggatgttccgcgcaatatcctgaccaaagcaccgtctgctggtctgtggcaaggccaaaccgatgaaggtgaaatgggcgttacctatcaggaaattgataattttctggacggtaaagaagtatcgccggcaacctttgaaaaaattagctactggcataatcgctctcaccacaaacgcaaaatggctctgacgccggattttaactaa

SEQ ID NO: 34 - DNA, 750 bp, Francisella persica
  misc_feature (1)..(750): Optimized_CP013022 Francisella persica ATCC VR-331, NadE
atgaaaattgttaaagatttcaacatcaaagaatattcacaaaaactgattgattggctgagtgatacctgtatgaattacccggctgaaggctttgtcattggtctgagcggtggtattgattcggcagttgcagcttctctggctgtcaaaaccggcctgtcaaccaccgctctgattctgccgtcaaaaaacaatcaacaccaagatattcaagatgctctggaactggcagataaaattaatattgaacatcataccattaccattcaaaccgtatacgaaacctttctggcgtcaattaaaaaaattaccaataccgaacgtgatcgccaactggtcattaaaggcaatgctcaagctcgtctgcgcatgatgtatctgtatgcctatgctcaacaatataatcgcgtggttattggtaccgataatgcttgtgaatggtatatgggctattttaccaaatttggtgatggtgctgctgatattctgccgctggttaatctgaaaaaatctcacgtttttgaactgggtaaatacctgggtgttccgaaaaatattctggataaagctccgtctgctggcctgtggcaaggccaaaccgatgaagatgaaatgggcgtaacctatcaagaaattgatgatttcctggatggtaaacaagtttcagcgaaagctctggaacgcattaatttctggcataatcgtagtcatcataaacgcaaactggctctgattccgaatttctaa

SEQ ID NO: 35 - DNA, 750 bp, Unknown
  FtNadE_star_optimized
  misc_feature (1)..(750): FtNadE_star_optimized
atgaaaatcgtcaaagacttctccccgaaagaatattcccaaaaactggtgaactggctgagcgactcgtgtatgaactatccggcagaaggctttgtcattggtctgagtggcggtatcgattccgctgtggcggcctcactggccgttaaaaccggcctgccgaccacggcactgattctgccgtctgacaacaatcagcatcaagatatgcaggacgcgctggaactgattgaaatgctgaacatcgaacactacaccatttccatccagccggcgtatgaagcgtttctggcgagcacccaatctttcacgaacctgcagaacaatcgtcaactggtgatcaaaggcaatgcgcaggcccgtctgcgcatgatgtatctgtacgcgtatgcgcagcaatacaaccgcattgttatcggcaccgataatgcctgcgaatggtacatgggttattttacgaaattcggcgatggtgcagctgacattctgccgctggtcaacctgaaaaaatcgcaggtgtttgaactgggtaaatacctggatgttccgaaaaatatcctggacaaagcaccgagcgcaggtctgtggcagggtcaaaccgatgaagacgaaatgggcgttacgtatcaggaaattgatgacttcctggatggtaaacaagtcagcgcgaaagccctggaacgtatcaacttctggcacaaccgctcacatcataaacgcaaactggcactgaccccgaacttctaa

SEQ ID NO: 36 - DNA, 782 bp, Bacillus
  misc_feature (1)..(782): Optimized_FtNadE_star_Bacillus
gctacctgagaagcttatgaaaatcgttaaagacttctctccgaaagaatattctcaaaaacttgtgaactggcttagcgactcttgtatgaactatccggcagaaggctttgttattggtctttctggcggtatcgactctgctgtggcggcttcacttgctgttaaaacaggtcttccgacaacagcacttattcttccgtctgacaacaatcagcatcaagatatgcaggacgcgcttgaacttattgaaatgcttaacatcgaacactacacaatttctatccagccggcgtatgaagcgtttcttgcgagcacacaatctttcacaaaccttcagaacaatcgtcaacttgtgatcaaaggcaatgcgcaggctcgtcttcgcatgatgtatctttacgcgtatgcgcagcaatacaaccgcattgttatcggcacagataatgcttgcgaatggtacatgggttattttacaaaattcggcgatggtgctgctgacattcttccgcttgttaaccttaaaaaatctcaggtgtttgaacttggtaaataccttgatgttccgaaaaatatccttgacaaagcaccgagcgcaggtctttggcagggtcaaacagatgaagacgaaatgggcgttacatatcaggaaattgatgacttccttgatggtaaacaagttagcgcgaaagctcttgaacgtatcaacttctggcacaaccgctcacatcataaacgcaaacttgcacttacaccgaacttctaagcatgcagtaagtagc

SEQ ID NO: 37 - DNA, 785 bp, Bacillus Mannheimia succiniciproducens
  misc_feature (1)..(785): Optimized_AE016827_Bacillus Mannheimia succiniciproducens MBEL55E, complete genome
gctacctgagaagcttatgaaaacagcagcatacgcagattatcttattcaatggcttgaaaaccaacgcacagaactttacggcatggacggctatacacttggcgttagcggcggtattgacagcgctgtttgcgctcatcttgcagcgcgcacaggcgcgccggtacaagctcttattcttccggcggaagtaacatctccgtcagatgtggcggatgcgcaagctacacttgaaagcgctggtattgacggccaaattatttctattgcaccgtggtacgatcttattatgcaacaactttctccggtacttaatagcgaaccggaacgcgttaacgtacttaaaggtaatcttatggcacgccttcgtatgattgcgctttttacaacagcacaaagccatcgttctattgtgcttggcacagataatgcggcggaatggcttacaggttattttacaaaattcggcgacggcgcagcggacgtacttccgcttgcgggccttcgcaaagaacaggtatttgaacttggccgttatcttggcgtaccgcaaagcgtgcttgataaaaaaccgagcgctggtctttgggcaggccaaacagacgaagctgaaatgggtgttacatacgcggaaatcgacgcttatcttcgcggcgaaacagttagcccgcaggcacttcaacaaatccgtttctggcacaaccgttctcatcacaaacgtatgcttccgccgaaaccgaaatcaccggatgaagcggaatgttaagcatgcagtaagtagc

SEQ ID NO: 38 - DNA, 782 bp, Bacillus Francisella
  misc_feature (1)..(782): Optimized_CP002558_Bacillus Francisella cf. novicida 3523, complete genome
gctacctgagaagcttatgaaaattgttaaagatttttctccgaaagaatattcacaaaatcttgttaattggctttctgatacatgtattaattatccggctgaaggctttgtaatcggcattagcggtggtattgattcagcggttgcagcttctcttgctgttaaaacaggccttccgacaacagctcttattcttccgtcaaaaaacaatcaacaccaagatattcaagatgctcttgaacttgttgaaaaacttaatattgaacatcatattgttacaattcaaccggcatacgaaaattttcttgcatcaacacaggaatttattaatacagataataatcgccaacttgtgatcaaaggcaatgctcaagcacgtcttcgcatgatgtatctttatgcttatgctcaacaatataaccgcattgttattggtacagataatgcttgtgaatggtatatgggctattttacaaaatttggtgatggcgctgctgatatttttccgcttattaatcttaaaaaatcacaagtttttgaacttggtaaataccttgatgttccgaaaaatattattgataaagctccgtctgctggcctttggcaaggccaaacagatgaagatgaaatgggcgtaacatatcaagaaattgatgatttccttgatggtaaacaaatttcagcaaaagctcttgaacgcattaacttctggcataatcgttctcatcataaacgcaaacttgctcttacaccgaatttctaagcatgcagtaagtagc

SEQ ID NO: 39 - DNA, 782 bp, Francisella sp.
  misc_feature (1)..(782): Optimized_CP002872_Bacillus Francisella sp. TX077308, complete genome
gctacctgagaagcttatgaaaattgtaaaaaactttattgtagaacagtattctaataatcttattaaatggcttaaagaaaattgcattaaatatccggctgaaggttttgtgattggtatttctggtggtattgattctgcagtagctgcatctcttgcagttaaaacaggccttccgacaacagctcttattcttccgtctaaaaacaatcaagaccaagatatgcgcgatggcattgaacttatcgaaaatcttaatattgaatatcatacagtttcaattcaaccggcttatgacacatttattgaatcaacatttaactttacaaactcacaaaatgatcgccaacatgttatcaaaggcaatgctcaagcgcgtcttcgcatgatgtatctttatgcttatgctcagcaaaataatcgcattgttattggtacagataacgcatgtgaatggtacatgggctatttcacaaaatttggtgatggtgcagcagatattcttccgcttattaatcttaaaaaatctcaagtttttgaacttggtaaataccttaaagtgccgaaaaacattatccaaaaagatccgtctgctggtctttggcaaggtcaaacagatgaagatgaaatgggtgttacatacaaagaaattgatgacttccttgacggtaaagaagtttcagaaaaagctcttgaacgcattagcttctggcataatcgttctcaccataaacgcagcatggcttttacaccgaatttttaagcatgcagtaagtagc

SEQ ID NO: 40 - DNA, 750 bp, Unknown
  DQ682092-AL_Reverted
  misc_feature (1)..(750): DQ682092-AL_Reverted
atgaaaatcgtcaaagacttctccccgaaagaatattcccaaaaactggtgaactggctgagcgactcgtgtatgaacactccggcagaaggctttgtcattggtctgagtggcggtatcgattccgctgtggcggcctcactggccgttaaaaccggcctgccgaccacggcactgattctgccgtctgacaacaatcagcatcaagatatgcaggacgcgctggaactgattgaaatgctgaacatcgaacactacaccatttccatccagccggcgtatgaagcgtttctggcgagcacccaatctttcacgaacctgcagaacaatcgtcaactggtgatcaaaggcaatgcgcaggcccgtctgcgcatgatgtatctgtacgcgtatgcgggtcaatacaaccgcattgttatcggcaccgataatgcctgcgaatggtacatgggttattttacgaaattcggcgatggtgcagctgacattctgccgctggtcaacctgaaaaaatcgcaggtgtttgaactgggtaaatacctggatgttccgaaaaatatcctggacaaagcaccgagcgcaggtctgtggcagggtcaaaccgatgaagacgaaatgggcgttacgtatcaggaaattgatgacttcctggatggtaaacaagtcagcgcgaaagccctggaacgtatcaacttctggcacaacgtctcacatcataaacgcaaactggcactgaccccgaacttctaa

SEQ ID NO: 41 - protein, 540 aa, Escherichia coli
  misc_feature (1)..(540): Escherichia coli NadB
MNTLPEHSCDVLIIGSGAAGLSLALRLADQHQVIVLSKGPVTEGSTFYAQGGIAAVFDETDSIDSHVEDTLIAGAGICDRHAVEFVASNARSCVQWLIDQGVLFDTHIQPNGEESYHLTREGGHSHRRILHAADATGREVETTLVSKALNHPNIRVLERSNAVDLIVSDKIGLPGTRRVVGAWVWNRNKETVETCHAKAVVLATGGASKVYQYTTNPDISSGDGIAMAWRAGCRVANLEFNQFHPTALYHPQARNFLLTEALRGEGAYLKRPDGTRFMPDFDERGELAPRDIVARAIDHEMKRLGADCMFLDISHKPADFIRQHFPMIYEKLLGLGIDLTQEPVPIVPAAHYTCGGVMVDDHGRTDVEGLYAIGEVSYTGLHGANRMASNSLLECLVYGWSAAEDITRRMPYAHDISTLPPWDESRVENPDERVVIQHNWHELRLFMWDYVGIVRTTKRLERALRRITMLQQEIDEYYAHFRVSNNLLELRNLVQVAELIVRCAMMRKESRGLHFTLDYPELLTHSGPSILSPGNHYINR

SEQ ID NO: 42 - protein, 531 aa, Bacillus subtilis
  misc_feature (1)..(531): Bacillus subtilis NadB
MSKKTIAVIGSGAAALSLAAAFPPSYEVTVITKKSVKNSNSVYAQGGIAAAYAKDDSIEAHLEDTLYAGCGHNNLAIVADVLHDGKMMVQSLLERGFPFDRNERGGVCLGREGAHSYNRIFHAGGDATGRLLIDYLLKRINSKIKLIENETAADLLIEDGRCIGVMTKDSKGRLKVRHADEVVLAAGGCGNLFLHHTNDLTVTGDGLSLAYRAGAELTDLEFTQFHPTLLVKNGVSYGLVSEAVRGEGGCLVDENGRRIMAERHPLGDLAPRDIVSRVIHEEMAKGNRVYIDFSAISDFETRFPTITAICEKAGIDIHSGKIPVAPGMHFLMGGVSVNRWGETTVPGLYAIGETACSGLHGANRLASNSLLEALVFGKRAAEHIIQKPVYNRQYQSGLETSVFYEVPDIEGHELQSKMTSHMSILREQSSLIELSIWLHTLPFQEVNVKDITIRQMELSHLWQTAKLMTFSALLREESRGAHFRTDFPHAEVSWQGRQIVHTKKGTKIRKNEGIWNNESFTAEKITESLFS

SEQ ID NO: 43 - DNA, 750 bp, Francisella sp.
  misc_feature (1)..(750): Optimized_CP002872_Reverted Francisella sp. TX077308
atgaaaattgtaaaaaactttattgtagaacagtattctaataatctgattaaatggctgaaagagaattgcattaaaactccggctgaaggttttgtgattggtattagtggtggtattgattcggcagtagccgcatctctggcagtcaaaaccggcctgccgaccaccgctctgattctgccgtcgaaaaacaatcaagaccaagatatgcgcgatggcattgaactgatcgaaaatctgaatattgagtatcataccgtttcaattcaaccggcttatgacacgtttattgagtcaacctttaactttaccaactcacaaaatgatcgccaacatgttatcaaaggcaatgcccaagcgcgtctgcgcatgatgtatctgtatgcttatgctgggcaaaataatcgcattgttattggtaccgataacgcatgtgaatggtacatgggctatttcaccaaatttggtgatggtgcagcagatattctgccgctgattaatctgaaaaaatctcaagtttttgaactgggtaaatacctgaaagtgccgaaaaacattatccaaaaagatccgtctgccggtctgtggcaaggtcaaaccgatgaggatgaaatgggtgtcacctacaaagaaattgatgacttcctggacggtaaagaagtctcagaaaaagctctggaacgcattagcttctggcataatgttagtcaccataaacgcagcatggcttttaccccgaatttttaa

SEQ ID NO: 44 - DNA, 828 bp, E. coli
  misc_feature (1)..(828): E. coli NadE
atgacattgcaacaacaaataataaaggcgctgggcgcaaaaccgcagattaatgctgaagaggaaattcgtcgtagtgtcgattttctgaaaagctacctgcaaacttatccgttcattaaatcactggtgctcgggatcagcggcggtcaggactccacgcttgccggaaagctgtgccagatggcgattaatgagctgcgcctggaaaccggcaacgaatcactgcaatttattgccgtacgcctgccctatggtgttcaggccgacgaacaagattgccaggatgccattgcctttattcaaccggatcgcgtattaaccgttaatatcaagggcgcggtattggccagcgaacaggcattgcgggaagcaggcattgaactgagcgattttgtccgtggcaatgaaaaagcgcgtgagcggatgaaagcacaatatagcattgcgggtatgaccagcggtgtcgtggtgggcaccgatcatgcagcagaagccattaccggattcttcactaaatatggtgacggcggtacggacattaatccgctgtatcgtctcaacaaacgtcagggtaaacagttactggcggcattagcttgcccggaacacctttataagaaagcgccaacggccgatctggaagatgatcgcccttctctgccagatgaagtggcactcggcgtgacctatgacaatatcgacgactatctggaagggaaaaacgtacctcaacaggtcgccagaacaatagagaactggtatctgaaaaccgaacataaacgccgtccgccaattaccgttttcgatgatttctggaaaaagtaa

SEQ ID NO: 45 - DNA, 750 bp, Unknown
  C. glutamicum
  misc_feature (1)..(750): FnNadE optimized for C. glutamicum
atgaaaattgtgaaagatttttccccaaaagaatattcccaaaatctggtgaattggctgtccgatacctgtattaattatccagcagaaggctttgtgatcggcatttccggtggtattgattccgcagtggcagcatccctggcagtgaaaaccggcctgccaaccaccgcactgattctgccatccaaaaacaatcaacaccaagatattcaagatgcactggaactggtggaaaaactgaatattgaacatcatattgtgaccattcaaccagcatacgaaaattttctggcatccacccaggaatttattaataccgataataatcgccaactggtgatcaaaggcaatgcacaagcacgcctgcgcatgatgtatctgtatgcatatgcacaacaatataaccgcattgtgattggtaccgataatgcatgtgaatggtatatgggctattttaccaaatttggtgatggcgcagcagatatttttccactgattaatctgaaaaaatcccaagtgtttgaactgggtaaatacctggatgtgccaaaaaatattattgataaagcaccatccgcaggcctgtggcaaggccaaaccgatgaagatgaaatgggcgtgacctatcaagaaattgatgatttcctggatggtaaacaaatttccgcaaaagcactggaacgcattaacttctggcataatcgctcccatcataaacgcaaactggcactgaccccaaatttctaa

SEQ ID NO: 46 - DNA, 828 bp, E. coli; identical to SEQ ID NO: 44
  misc_feature (1)..(828): E. coli NadE enzyme (EcNadE)

SEQ ID NO: 47 - DNA, 750 bp, Francisella
  misc_feature (1)..(750): Optimized_CP002558_Reverted Francisella cf. novicida 3523
atgaaaattgttaaagattttagtccgaaagaatattcacaaaatctggttaattggctgagtgatacctgtattaatactccggctgaaggctttgtaatcggcattagcggtggtattgattcagcggttgcagcttctctggctgtcaaaaccggcctgccgaccaccgctctgattctgccgtcaaaaaacaatcaacaccaagatattcaagatgctctggaactggttgagaaactgaatattgaacatcatattgttaccattcaaccggcatacgaaaattttctggcatcaacccaggaatttattaataccgataataatcgccaactggtgatcaaaggcaatgctcaagcacgtctgcgcatgatgtatctgtatgcctatgccggccaatataaccgcattgttattggtaccgataatgcttgtgagtggtatatgggctattttaccaaatttggtgatggcgctgctgatatttttccgctgattaatctgaaaaaatcacaagtttttgaactgggtaaatacctggatgttccgaaaaatattattgataaagctccgtctgctggcctgtggcaaggccaaaccgatgaggatgaaatgggcgtaacctatcaagaaattgatgatttcctggatggtaaacaaatttcagcaaaagccctggaacgcattaacttctggcataatgttagtcatcataaacgcaaactggctctgaccccgaatttctaa

SEQ ID NO: 48 - protein, 349 aa, Unknown
  CAG83794 - NMN nucleotidase
  misc_feature (1)..(349): CAG83794 - NMN nucleotidase
MKFSNLALPLLASCAVAQNSTNPNSNTTIIVTNDDSWASANIRSLYSELKKEGYNVFMFAPAVQQSGTGGTFNLPRAANLTKGGEFGSIPVGAPNWGQDDNDDHIWYFDGTPAAAMSFGLDYAIPRLFNNATVDLVVSGPNEGWNIGPNIYTMSGTNGAMYMAVLRGIPAIAYSGMNSHQYYANASTSDNAAHNIYAKAATGIVNNLVKNAKDRATLLPYGVGLSVNLPRAGDVDPTGQCKEIKPIFTRQTGPAAIVLKLSYNETTNRFSPGITNSEASKACLNGDCILPDESDVVANWGCYASVSVITTDYDAPRDVAGEVQFLNRGEVKFAPRVYGSFAPVEASPYF

SEQ ID NO: 49 - protein, 271 aa, Unknown
  An02g02380 - NMN nucleotidase (SDT1 homolog)
  misc_feature (1)..(271): An02g02380 - NMN nucleotidase (SDT1 homolog)
MTTTSSPASAPLDPRPVFFFDIDNCLYSKGCNIHDEMQKLINQFFIKHLSLNADDAHMLHMKYYKEYGLAIEGLTRHHKIDPLEFNREVDDALPLDDILKPDPKLRRLLEDIDRSKVRMWLLTNAYVTHAKRVVKLLQVDDLFEGITYCDYGNSPLVCKPSQAMYERAEKEAGASSTSECYFVGKSPSQFLSHSTYPDQVLTDRSRRLRPELYSCCRPGLGCGSSCRAWNTPPPRAGLAIYDTKPGGTQDLFPHLIQDEAGSIDSITLCDR

SEQ ID NO: 50 - protein, 411 aa, Unknown
  An02g02380 - NMN nucleotidase (SDT1 homolog)
  misc_feature (1)..(411): An02g02380 - NMN nucleotidase (SDT1 homolog)
MTTRYRVEYALKSHRRDQLIEWIKGLLAVPFVLHSQPTAVYQEHSENLIAVAADTHQRYAEIFRDVEMLIRDHITHQQDDAPGKSKLKLLVPTVGTFFTPLYLVDAFRRQDAQRFISRRRFVAPSFNDIRLILNSAQLIGLARTTGVDLVTFDGDVTLYDDGACLTDDNPVILRIMRLLLQGRKVGIVTAAGYTDAAKYYERLKGLLDAMYESAEMTDAQRAGLVVMGGESNFLFRYDHASPSRLSYVPREEWLLEEMKTWQEGDITRLLDIAESSLRACASNLNLPVAVLRKDRAVGVYPTERGRISREQLEETVLVVQNTVERSEVGARLPFCAFNGGNDVFVDIGDKSWGVRACQRYFGGIEPARTLHVGDQFLSAGANDFKARLASTTAWIASPAETVQLLDELQGI

SEQ ID NO: 51 - protein, 410 aa, Escherichia coli
  misc_feature (1)..(410): Escherichia coli NadR (NMN synthetase, NR kinase, negative regulator of NAD biosynthesis) >gi|90111746|ref|NP_418807.4| trifunctional protein nicotinamide mononucleotide adenylyltransferase, ribosylnicotinamide kinase, transcriptional
MSSFDYLKTAIKQQGCTLQQVADASGMTKGYLSQLLNAKIKSPSAQKLEALHRFLGLEFPRQKKTIGVVFGKFYPLHTGHIYLIQRACSQVDELHIIMGFDDTRDRALFEDSAMSQQPTVPDRLRWLLQTFKYQKNIRIHAFNEEGMEPYPHGWDVWSNGIKKFMAEKGIQPDLIYTSEEADAPQYMEHLGIETVLVDPKRTFMSISGAQIRENPFRYWEYIPTEVKPFFVRTVAILGGESSGKSTLVNKLANIFNTTSAWEYGRDYVFSHLGGDEIALQYSDYDKIALGHAQYIDFAVKYANKVAFIDTDFVTTQAFCKKYEGREHPFVQALIDEYRFDLVILLENNTPWVADGLRSLGSSVDRKEFQNLLVEMLEENNIEFVRVEEEDYDSRFLRCVELVREMMGEQR

SEQ ID NO: 52 - protein, 180 aa, Bacillus subtilis
  misc_feature (1)..(180): Bacillus subtilis YxrA (repressor protein) >gi|251757401|sp|P39667.2|NADR_BACSU RecName Full=Transcription repressor NadR; AltName Full=ORF1
MTEELKLMGANRRDQLLLWLKESKSPLTGGELAKKANVSRQVIVQDISLLKAKNVPIIATSQGYVYMDAAAQQHQQAERIIACLHGPERTEEELQLIVDEGVTVKDVKIEHPVYGDLTAAIQVGTRKEVSHFIKKINSTNAAYLSQLTDGVHLHTLTAPDEHRIDQACQALEEAGILIKD

SEQ ID NO: 53 - protein, 214 aa, Corynebacterium glutamicum
  misc_feature (1)..(214): Corynebacterium glutamicum CgR_1153 (repressor protein) >gi|140845132|dbj|BAF54131.1| hypothetical protein cgR_1153 [Corynebacterium glutamicum R]
MPASPEIQMAVSTIIFALRPGPQDLPSLWAPFVPRTREPHLNKWALPGGWLPPHEELEDAAARTLAETTGLHPSYLEQLYTFGKVDRSPTGRVISVVYWALVRADEALKAIPGENVQWFPADHLPELAFDHNDIVKYALERLRTKVEYSEIAHSFLGETFTIAQLRSVHEAVLGHKLDAANFRRSVATSPDLIDTGEVLAGTPHRPPKLFRFQR

SEQ ID NO: 54 - protein, 191 aa, Acinetobacter baylyi
  misc_feature (1)..(191): Acinetobacter baylyi PnuC (NR transporter protein) >gi|49530211|emb|CAG67923.1| putative nucleoside/purine/pyrimidine transport protein (NMN family) (PnuC) [Acinetobacter sp. ADP1]
MSPLEIFAVIISVIGVALTIKRNMWCWGFNFLAFILYGYLFFSFKLYGETILQGFFIIINFYGFYYWLKGKQTEHEIRIVAIPAKTVIIQMLLAALGGLIFGLSLKHFTDAAVPMLDSQLAAFSLLATYWTSRKHIATWVLWVFVDIVYVGMFIYKDLYLTAGLYAAFVVMAAFGWWQWEQVKRKQRSGLI

SEQ ID NO: 55 - protein, 230 aa, Corynebacterium glutamicum
  misc_feature (1)..(230): Corynebacterium glutamicum NR transporter protein >gi|19551314|ref|NP_599316.1| nicotinamide mononucleotide transporter [Corynebacterium glutamicum ATCC 13032]
MNPITELLDATLWIGGVPILWREIIGNVFGLFSAWAGMRRIVWAWPIGIIGNALLFTVFMGGLFHTPQNLDLYGQAGRQIMFIIVSGYGWYQWSAAKRRALTPENAVAVVPRWASTKERAGIVIAAVVGTLSFAWIFQALGSWGPWADAWIFVGSILATYGMARGWTEFWLIWIAVDIVGVPLLLTAGYYPSAVLYLVYGAFVSWGFVVWLRVQKADKARALEAQESVTV

SEQ ID NO: 56 - protein, 239 aa, Escherichia coli
  misc_feature (1)..(239): Escherichia coli PnuC (NR transporter protein) >gi|16128719|ref|NP_415272.1| nicotinamide riboside transporter [Escherichia coli str. K-12 substr. MG1655]
MDFFSVQNILVHIPIGAGGYDLSWIEAVGTIAGLLCIGLASLEKISNYFFGLINVTLFGIIFFQIQLYASLLLQVFFFAANIYGWYAWSRQTSQNEAELKIRWLPLPKALSWLAVCVVSIGLMTVFINPVFAFLTRVAVMIMQALGLQVVMPELQPDAFPFWDSCMMVLSIVAMILMTRKYVENWLLWVIINVISVVIFALQGVYAMSLEYIILTFIALNGSRMWINSARERGSRALSH

SEQ ID NO: 57 - protein, 550 aa, Escherichia coli
  misc_feature (1)..(550): Escherichia coli UshA (nucleotide hydrolase) >gi|16128464|ref|NP_415013.1| bifunctional UDP-sugar hydrolase/5′-nucleotidase [Escherichia coli str. K-12 substr. MG1655]
MKLLQRGVALALLTTFTLASETALAYEQDKTYKITVLHTNDHHGHFWRNEYGEYGLAAQKTLVDGIRKEVAAEGGSVLLLSGGDINTGVPESDLQDAEPDFRGMNLVGYDAMAIGNHEFDNPLTVLRQQEKWAKFPLLSANIYQKSTGERLFKPWALFKRQDLKIAVIGLTTDDTAKIGNPEYFTDIEFRKPADEAKLVIQELQQTEKPDIIIAATHMGHYDNGEHGSNAPGDVEMARALPAGSLAMIVGGHSQDPVCMAAENKKQVDYVPGTPCKPDQQNGIWIVQAHEWGKYVGRADFEFRNGEMKMVNYQLIPVNLKKKVTWEDGKSERVLYTPEIAENQQMISLLSPFQNKGKAQLEVKIGETNGRLEGDRDKVRFVQTNMGRLILAAQMDRTGADFAVMSGGGIRDSIEAGDISYKNVLKVQPFGNVVVYADMTGKEVIDYLTAVAQMKPDSGAYPQFANVSFVAKDGKLNDLKIKGEPVDPAKTYRMATLNFNATGGDGYPRLDNKPGYVNTGFIDAEVLKAYIQKSSPLDVSVYEPKGEVSWQ

SEQ ID NO: 58 - protein, 1462 aa, Bacillus subtilis
  misc_feature (1)..(1462): Bacillus subtilis YfkN (nucleotide hydrolase) >gi|16077851|ref|NP_388665.1| nucleotide phosphoesterase [Bacillus subtilis subsp. subtilis str. 168]
MRIQKRRTHVENILRILLPPIMILSLILPTPPIHAEESAAPQVHLSILATTDIHANMMDYDYYSDKETADFGLARTAQLIQKHREQNPNTLLVDNGDLIQGNPLGEYAVKYQKDDIISGTKTHPIISVMNALKYDAGTLGNHEFNYGLDFLDGTIKGADFPIVNANVKTTSGENRYTPYVINEKTLIDENGNEQKVKVGYIGFVPPQIMTWDKKNLEGQVQVQDIVESANETIPKMKAEGADVIIALAHTGIEKQAQSSGAENAVFDLATKTKGIDAIISGHQHGLFPSAEYAGVAQFNVEKGTINGIPVVMPSSWGKYLGVIDLKLEKADGSWKVADSKGSIESIAGNVTSRNETVTNTIQQTHQNTLEYVRKPVGKTEADINSFFAQVKDDPSIQIVTDAQKWYAEKEMKDTEYKNLPILSAGAPFKAGGRNGANYYTNIPAGDLAIKNVGDLYLYDNTVQIVKLTGSEVKDWLEMSAGQFNQIDPAKGGDQALLNENFRSYNFDVIDGVTYQVDVTKPAKYNENGKVINADSSRIINLSYEGKPISPSQEFLVVTNNYRASGGGGFPHLTSDKIVHGSAVENRQVLMDYIIEQKTVNPKADNNWSIAPVSGTNLTFESSLLAKPFADKADDVAYVGKSANEGYGVYKLQFDDDSNPDPPKDGLWDLTVMHTNDTHAHLDDAARRMTKINEVRSETNHNILLDAGDVFSGDLYFTKWNGLADLKMMNMMGYDAMTFGNHEFDKGPTVLSDFLSGNSATVDPANRYHFEAPEFPIVSANVDVSNEPKLKSFVKKPQTFTAGEKKEAGIHPYILLDVDGEKVAVFGLTTEDTATTSSPGKSIVFNDAFETAQNTVKAIQEEEKVNKIIALTHIGHNRDLELAKKVKGIDLIIGGHTHTLVDKMEVVNNEEPTIVAQAKEYGQFLGRVDVAFDEKGVVQTDKSNLSVLPIDEHTEENPEAKQELDQFKNELEDVKNEKVGYTDVALDGQREHVRTKETNLGNFIADGMLAKAKEAAGARIAITNGGGIRAGIDKGDITLGEVLNVMPFGNTLYVADLTGKQIKEALEQGLSNVENGGGAFPQVAGIEYTFTLNNKPGHRVLEVKIESPNGDKVAINTDDTYRVATNNFVGAGGDGYSVFTEASHGEDLGYVDYEIFTEQLKKLGNKVSPKVEGRIKEVFLPTKQKDGSWTLDEDKFAIYAKNANTPFVYYGIHEGSQEKPINLKVKKDQVKLLKERESDPSLTMFNYWYSMKMPMANLKTADTAIGIKSTGELDVSLSDVYDFTVKQKGKEIKSFKEPVQLSLRMFDIEEAHNPAIYHVDRKKKAFTKTGHGSVDDDMVTGYTNHFSEYTILNSGSNNKPPAFPSDQPTGGDDGNHGGGSDKPGGKQPTDGNGGNDTPPGTQPTNGSGGNGSGGSGTDGPAGGLLPDTATSMYSILLAGFLISALGTAMYLHQRRKQNRANQA

SEQ ID NO: 59 - protein, 694 aa, Corynebacterium glutamicum
  misc_feature (1)..(694): Corynebacterium glutamicum Cg0397 (nucleotide hydrolase) >gi|41324559|emb|CAF18899.1| PROBABLE 5′-NUCLEOTIDASE PRECURSOR [Corynebacterium glutamicum ATCC 13032]
MKRLSRAALAVVATTAVSFSALAVPAFADEASNVELNILGVTDFHGHIEQKAVKDDKGVITGYSEMGASGVACYVDAERADNPNTRFITVGDNIGGSPFVSSILKDEPTLQALSAIGVDASALGNHEFDQGYSDLVNRVSLDGSGSAKFPYLGANVEGGTPAPAKSEIIEMDGVKIAYVGAVTEETATLVSPAGIEGITFTGDIDAINAEADRVIEAGEADVVIALIHAEAAPTDLFSNNVDVVFSGHTHFDYVAEGEARGDKQPLVVIQGHEYGKVISDVEISYDREAGKITNIEAKNVSATDVVENCETPNTAVDAIVAAAVEAAEEAGNEVVATIDNGFYRGADEEGTTGSNRGVESSLSNLIAEAGLWAVNDATILNADIGIMNAGGVRADLEAGEVTFADAYATQNFSNTYGVREVSGAQFKEALEQQWKETGDRPRLALGLSSNVQYSYDETREYGDRITHITFNGEPMDMKETYRVTGSSFLLAGGDSFTAFAEGGPIAETGMVDIDLFNNYIAAHPDAPIRANQSSVGIALSGPAVAEDGTLVPGEELTVDLSSLSYTGPEAKPTTVEVTVGTEKKTADVDNTIVPQFDSTGKATVTLTVPEGATSVKIATDNGTTFELPVTVNGEGNNDDDDDKEQQSSGSSDAGSLVAVLGVLGALGGLVAFFLNSAQGAPFLAQLQAMFAQFM

SEQ ID NO: 60 - protein, 165 aa, Escherichia coli
  misc_feature (1)..(165): Escherichia coli PncC (nicotinamide mononucleotide amidohydrolase) >gi|16130607|ref|NP_417180.1| nicotinamide-nucleotide amidohydrolase; NMN amidohydrolase [Escherichia coli str. K-12 substr. MG1655]
MTDSELMQLSEQVGQALKARGATVTTAESCTGGWVAKVITDIAGSSAWFERGFVTYSNEAKAQMIGVREETLAQHGAVSEPVVVEMAIGALKAARADYAVSISGIAGPDGGSEEKPVGTVWFAFATARGEGITRRECFSGDRDAVRRQATAYALQTLWQQFLQNT

SEQ ID NO: 61 - protein, 416 aa, Bacillus subtilis
  misc_feature (1)..(416): Bacillus subtilis CinA (nicotinamide mononucleotide amidohydrolase) >gi|1314847|gb|AAB00568.1| CinA [Bacillus subtilis subsp. subtilis str. 168]
MEFPKKAEIIAVGSELLLGQIANTNAQFISKQLAEIGVHVFYHTAVGDNPERLKQVIRIAEERSDFIIFSGGLGPTKDDLTKETIANTLGRPLVLNDEAFQSIEDYPKRTKRTMSPNNRKQALVIEGSDVLANHFGMAPGMLTEHESRYYMLLPGPPSELRPMFENEAKPLLLKKMGSNEKIVSTVLRFFGIGESQLEPDLEDIIDAQTNPTIAPLAADGEVTLRLTAKHADEKETERLLKETEAVILERVGEFFYGYDDTSLVKELSIACKEKGITISAAESFTGGLFSEWLTDHSGASKLFAGGVVCYTNDVKQNVLGVKKETLDRFGAVSKECASELAKGVQKLTGSDIGISFTGVAGPDAQEGHEPGHVFIGISANGKEEVHEFHFAGSRTGIRKRGAKYGCHLILKLLEQK

SEQ ID NO: 62 - protein, 172 aa, Corynebacterium glutamicum
  misc_feature (1)..(172): Corynebacterium glutamicum  Cg2153 (nicotinamide mononucleotide amidohydrolase) >gi|41326141|emb|CAF20304.1| similar to competence-and mitomycin-induced protein [Corynebacterium glutamicum ATCC 13032]
MSENLAGRVVELLKSRGETLAFCESLTAGLASATIAEIPGASVVLKGGLVTYATELKVALAGVPQELIDAHGVVSPQCARAMATGAAHRCQADWAVSLTGVAGPSKQDGHPVGEVWIGVAGPAHFGASGTIDAYRAFESEQQVILAELGRHHIRESAVQQSFRLLIDHIESQ

SEQ ID NO: 63 - protein, 213 aa, Escherichia coli
  misc_feature (1)..(213): Escherichia coli NadD (nicotinic acid mononucleotide adenyltransferase) >gi|16128622|ref|NP_415172.1| nicotinic acid mononucleotide adenylyltransferase, NAD(P)-dependent [Escherichia coli str. K-12 substr. MG1655]
MKSLQALFGGTFDPVHYGHLKPVETLANLIGLTRVTIIPNNVPPHRPQPEANSVQRKHMLELAIADKPLFTLDERELKRNAPSYTAQTLKEWRQEQGPDVPLAFIIGQDSLLTFPTWYEYETILDNAHLIVCRRPGYPLEMAQPQYQQWLEDHLTHNPEDLHLQPAGKIYLAETPWFNISATIIRERLQNGESCEDLLPEPVLTYINQQGLYR

SEQ ID NO: 64 - protein, 189 aa, Bacillus subtilis
  misc_feature (1)..(189): Bacillus subtilis NadD (nicotinic acid mononucleotide adenyltransferase) >gi|16079618|ref|NP_390442.1| nicotinate-nucleotide adenylyltransferase [Bacillus subtilis subsp. subtilis str. 168]
MKKIGIFGGTFDPPHNGHLLMANEVLYQAGLDEIWFMPNQIPPHKQNEDYTDSFHRVEMLKLAIQSNPSFKLELVEMEREGPSYTFDTVSLLKQRYPNDQLFFIIGADMIEYLPKWYKLDELLNLIQFIGVKRPGFHVETPYPLLFADVPEFEVSSTMIRERFKSKKPTDYLIPDKVKKYVEENGLYES

SEQ ID NO: 65 - protein, 226 aa, Corynebacterium glutamicum
  misc_feature (1)..(226): Corynebacterium glutamicum Cg2584 (nicotinic acid mononucleotide adenyltransferase) >gi|41326535|emb|CAF21017.1| PUTATIVE NICOTINIC ACID MONONUCLEOTIDE ADENYLYLTRANSFERASE [Corynebacterium glutamicum ATCC 13032]
MRTLYCPLMTTTVKRRARIGIMGGTFDPIHNGHLVAGSEVADRFDLDLVVYVPTGQPWQKANKKVSPAEDRYLMTVIATASNPRFMVSRVDIDRGGDTYTIDTLQDLSKQYPDAQLYFITGADALAQIVTWRDWEKTFELAHFVGVTRPGYELDGNIIPEMHQDRVSLVDIPAMAISSTDCRERSSEERPVWYLVPDGVVQYIAKRQLYRPEGSDKDMDPKGQNQA

SEQ ID NO: 66 - protein, 338 aa, Unknown
  CAG77762 - NR hydrolase
  misc_feature (1)..(338): CAG77762 - NR hydrolase
MIPIWLDCDPGHDDAVAILLAAKLPAFNLLGISTVFGNAPLEKTTVNAMAVLKAIGQDSEIKVYPGAERIQSRGGGGDGGSHDQSNQSHSPSSCSRGHYETAPSTFAPSIHGESGLDGTSLLPEITCTIEPHVDSTLVAMANAIRNNPGTCLVATGSLTNVSALFDRFPDVKNLVETVSIMGGGFGMGNWTPYAEFNIWCDAESASQVLGDPIVAAKVVLCPLNVTHTAIATEEVLSQIKSVGGNVSQMFFELMTFFKETYESEFGFKDGPPVHDPLSVAVLLNGIVTKERRVTVDVTVGGEQRGMLTEQVGGHLTVVEEVDLNKFWALVVTCLEACK

SEQ ID NO: 67 - protein, 435 aa, Unknown
  An18g04340 - NR hydrolase (URH1 homolog)
  misc_feature (1)..(435): An18g04340 - NR hydrolase (URH1 homolog)
MTLCPFRIRKVGLRADIAPCPLIRLIGTGFSYLQQSPQSPFRWHLGLPSFFSFIPSRSGFTMDSSVDTPIPLWLDCDPGHDDAFAILLAAHHPSLKLLGITTIHGNASLENTTINATRVLEAIGRPEIPVYPGSKKPFCRPALHAPNIHGDSGLDGTDLLPKASTAPITDKNPILAMRDALMAHPKGTPWVVATGTLTNVALLFATFPEVAEHIAGLTIMGGGVGEGFTDAPMSRLKGQENRIGNVTPLAEFNIYCDPEASQAIFSDPVVASKTTLITLDLTHQVLADQSVRNLVRHGVDDASVQPTTLRQMLYELLIFFANTYETVFGLSTGPPLHDPLAVAVILSTLNPGYAKNHPDQVLKFDDRGGERFSVNVITDGLHGTDIALVGSLGRSVVEANPNAVCIPRGVDLDAFWNMINDCIKRADECNAARKA

SEQ ID NO: 68 - protein, 240 aa, Unknown
  Sc NRK1 - NR kinase
  misc_feature (1)..(240): Sc NRK1 - NR kinase
MTSKKVILVALSGCSSSGKTTIAKLTASLFTKATLIHEDDFYKHDNEVPVDAKYNIQNWDSPEALDFKLFGKELDVIKQTGKIATKLIHNNNVDDPFTKFHIDRQVWDELKAKYDSINDDKYEVVIVDGFMIFNNTGISKKFDLKILVRAPYEVLKKRRASRKGYQTLDSFWVDPPYYFDEFVYESYRANHAQLFVNGDVEGLLDPRKSKNIKEFINDDDTPIAKPLSWVCQEILKLCKD

SEQ ID NO: 69 - protein, 254 aa, Unknown
  CAG83148 - NR kinase
  misc_feature (1)..(254): CAG83148 - NR kinase
MIRRKIAEKKLAKEEDKREVTLVALSGPSSSGKSTLARLLRDILPHVIIIHQDDFYLEDSQIPVIDGVQDWDCPEAFDFKLLSKVLSHVKQTGELPKNFKSKEDQNSLGPAALDENAVDAFKRRMHPYMPEFENKLIVILDGIMVYHDAQFTELFDIKILVRSSYENLKSRREARSGYVTLEGFWKDPEGYFHNIVWPGYLKTHKQLFENENPNGEPSKDATREGIRIVPTTDFDVAETLDWVFDVILDYYDLE

SEQ ID NO: 70 - protein, 281 aa, Unknown
  An04g05800 - NR kinase (NRK1 homolog)
  misc_feature (1)..(281): An04g05800 - NR kinase (NRK1 homolog)
MTKSPLIPHPRDPTTQIQNWDTLSALDIPFLSASLSYIHAHGHLPPRLRSKEDLNDVAESGVSEEVVSRMREVVRGRVPSYFLEDNTNSVSGDGVRTIVFLEGFLLFAPPKEEDPRHGLRDVQEKMDVRLFLPARYDNVKERREGRSGYVTIGPAPVQQGGGGGTELPQRGSVEVDLEEEDDRPPQSFWEDPPGYVDDIVWPNYVQDHAWLLLPEGEEGSAQTAWKDADTQELVRLVGQGVNLRLDTGVTVAPGQGKASMTDILEWAVEEVLKHIAETEGQ

SEQ ID NO: 71 - protein, 311 aa, Unknown
  Sc PNP1 - NMN/purine phosphorylase
  misc_feature (1)..(311): Sc PNP1 - NMN/purine phosphorylase
MSDILNVSQQREAITKAAAYISAILEPHFKNTTNFEPPRTLIICGSGLGGISTKLSRDNPPPVTVPYQDIPGFKKSTVPGHSGTLMFGSMNGSPVVLMNGRLHGYEGNTLFETTFPIRVLNHMGHVRNLIVTNAAGGINAKYQACDLMCIYDHLNIPGLAGQHPLRGPNLDEDGPRFLALSDAYDLELRKLLFKKWKELKIQRPLHEGTYTFVSGPTFETRAESKMIRMLGGDAVGMSTVPEVIVARHCGWRVLALSLITNTCVVDSPASALDESPVPLEKGKATHAEVLENGKIASNDVQNLIAAVMGEL

SEQ ID NO: 72 - protein, 298 aa, Unknown
  CAG78849 - NMN/purine phosphorylase
  misc_feature (1)..(298): CAG78849 - NMN/purine phosphorylase
MTDTFDLSLKSANKISDKLPKELQNPQIGIVCGSGLGGLANALKAEPQVTIEYKDIPGFKVSTVAGHAGKLVVGLLGEKNVPVVCMVGRFHFYEGYDIQDTVFPIRVFSQIGIKTVILTNAAGGLNQDFKVGDIMLINDHINLPGLAGNNPLRGPNDEKFGVRFLPLSDAYDHDLRRAVYDIAKKQGVTRGIHEGTYAFVSGPTYESRAEARMLSTIGADAVGMSTVPEVIVARHCGIKVLALSLITNVVVLKKPDSALNDNAAKLDEGIADHSEVMEEGQRAADDLVGIVTDLVNVV

SEQ ID NO: 73 - protein, 433 aa, Bacillus subtilis
  misc_feature (1)..(433): Bacillus subtilis Pdp (nucleoside phosphorylase) >gi|50812307|ref|NP_391819.2| pyrimidine-nucleoside phosphorylase [Bacillus subtilis subsp. subtilis str. 168]
MRMVDIIIKKQNGKELTTEEIQFFVNGYTDGSIPDYQASALAMAIFFQDMSDRERADLTMAMVNSGETIDLSAIEGIKVDKHSTGGVGDTTTLVLAPLVAALDVPVAKMSGRGLGHTGGTIDKLEAIMGFHVELTKDEFIKLVNRDKVAVIGQSGNLTPADKKLYALRDVTGTVNSIPLIASSIMSKKIAAGADAIVLDVKTGAGAFMKTEEDAAELAKAMVRIGNNVGRQTMAVISDMSQPLGFAIGNALEVKEAIDTLKGEGPEDLHELVLTLGSQMVVLAKKADTLDEARAKLEEVMKNGKALEKFKDFLKNQGGDSSIVDDPSKLPQAAYQIDVPAKEAGVVSEIVADEIGVAAMLLGAGRATKEDEIDLAVGIMLRKKVGDKVEKGEPLVTLYANRENVDEVIAKVYDNIRIAAEAKAPKLIHTLITE

SEQ ID NO: 74 - protein, 271 aa, Bacillus subtilis
  misc_feature (1)..(271): Bacillus subtilis PupG (nucleoside phosphorylase) >gi|16079406|ref|NP_390230.1| purine nucleoside phosphorylase 1 [Bacillus subtilis subsp. subtilis str. 168]
MKDRIERAAAFIKQNLPESPKIGLILGSGLGILADEIENPVKLKYEDIPEFPVSTVEGHAGQLVLGTLEGVSVIAMQGRFHFYEGYSMEKVTFPVRVMKALGVEALIVTNAAGGVNTEFRAGDLMIITDHINFMGTNPLIGPNEADFGARFPDMSSAYDKDLSSLAEKIAKDLNIPIQKGVYTAVTGPSYETPAEVRFLRTMGSDAVGMSTVPEVIVANHAGMRVLGISCISNAAAGILDQPLSHDEVMEVTEKVKAGFLKLVKAIVAQYE

SEQ ID NO: 75 - protein, 694 aa, Corynebacterium glutamicum; identical to SEQ ID NO: 59
  misc_feature (1)..(694): Corynebacterium glutamicum Cg0397 (nucleotide hydrolase)

SEQ ID NO: 76 - protein, 347 aa, Escherichia coli
  misc_feature (1)..(347): Escherichia coli NadA >gi|260450103|gb|ACX40525.1| quinolinate synthetase complex, A subunit [Escherichia coli DH1]
MSVMFDPDTAIYPFPPKPTPLSIDEKAYYREKIKRLLKERNAVMVAHYYTDPEIQQLAEETGGCISDSLEMARFGAKHPASTLLVAGVRFMGETAKILSPEKTILMPTLQAECSLDLGCPVEEFNAFCDAHPDRTVVVYANTSAAVKARADWVVTSSIAVELIDHLDSLGEKIIWAPDKHLGRYVQKQTGGDILCWQGACIVHDEFKTQALTRLQEEYPDAAILVHPESPQAIVDMADAVGSTSQLIAAAKTLPHQRLIVATDRGIFYKMQQAVPDKELLEAPTAGEGATCRSCAHCPWMAMNGLQAIAEALEQEGSNHEVHVDERLRERALVPLNRMLDFAATLRG

SEQ ID NO: 77 - protein, 368 aa, Bacillus subtilis
  misc_feature (1)..(368): Bacillus subtilis NadA >gi|16079837|ref|NP_390663.1| quinolinate synthase A [Bacillus subtilis subsp. subtilis str. 168]
MSILDVIKQSNDMMPESYKELSRKDMETRVAAIKKKFGSRLFIPGHHYQKDEVIQFADQTGDSLQLAQVAEKNKEADYIVFCGVHFMAETADMLTSEQQTVVLPDMRAGCSMADMADMQQTNRAWKKLQHIFGDTIIPLTYVNSTAEIKAFVGKHGGATVTSSNAKKVLEWAFTQKKRILFLPDQHLGRNTAYDLGIALEDMAVWDPMKDELVAESGHTNVKVILWKGHCSVHEKFTTKNIHDMRERDPDIQIIVHPECSHEVVTLSDDNGSTKYIIDTINQAPAGSKWAIGTEMNLVQRIIHEHPDKQIESLNPDMCPCLTMNRIDLPHLLWSLEQIEKGEPSGVIKVPKAIQEDALLALNRMLSIT

SEQ ID NO: 78 - protein, 428 aa, Corynebacterium glutamicum
  misc_feature (1)..(428): Corynebacterium glutamicum NadA >gi|41325294|emb|CAF19774.1| QUINOLINATE SYNTHETASE [Corynebacterium glutamicum ATCC 13032]
MTTSITPSVNLALKNANSCNSELKDGPWFLDQPGMPDVYGPGASQNDPIPAHAPRQQVLPEEYQRASDDELHRRIREAKDTLGDKVVILGHFYQRDEVIQHADFVGDSFQLARAAKTRPEAEAIVFCGVHFMAETADLLSTDEQSVILPNLAAGCSMADMADLDSVEDCWEQLTSIYGDDTLIPVTYMNSSAALKGFVGEHGGIVCTSSNARSVLEWAFERGQRVLFFPDQHLGRNTAKAMGIGIDQMPLWNPNKPLGGNTVSELENAKVLLWHGFCSVHKRFTVEQINKARAEYPDVHVIVHPESPMPVVDAADSSGSTDFIVKAIQAAPAGSTFAIGTEINLVQRLAAQYPQHTIFCLDPVICPCSTMYRIHPGYLAWALEELVAGNVINQISVSESVAAPARVALERMLSVVPAAPVTPSSSKDA

SEQ ID NO: 79 - protein, 540 aa, Escherichia coli; identical to SEQ ID NO: 41
  misc_feature (1)..(540): Escherichia coli NadB >gi|260448346|gb|ACX38768.1| L-aspartate oxidase [Escherichia coli DH1]

SEQ ID NO: 80 - protein, 531 aa, Bacillus subtilis; identical to SEQ ID NO: 42
  misc_feature (1)..(531): Bacillus subtilis NadB >gi|16079839|ref|NP_390665.1| L-aspartate oxidase [Bacillus subtilis subsp. subtilis str. 168]

SEQ ID NO: 81 - protein, 297 aa, Escherichia coli
  misc_feature (1)..(297): Escherichia coli NadC >gi|260450686|gb|ACX41108.1| nicotinate-nucleotide pyrophosphorylase [Escherichia coli DH1]
MPPRRYNPDTRRDELLERINLDIPGAVAQALREDLGGTVDANNDITAKLLPENSRSHATVITRENGVFCGKRWVEEVFIQLAGDDVTIIWHVDDGDVINANQSLFELEGPSRVLLTGERTALNFVQTLSGVASKVRHYVELLEGTNTQLLDTRKTLPGLRSALKYAVLCGGGANHRLGLSDAFLIKENHIIASGSVRQAVEKASWLHPDAPVEVEVENLEELDEALKAGADIIMLDNFETEQMREAVKRTNGKALLEVSGNVTDKTLREFAETGVDFISVGALTKHVQALDLSMRFR

SEQ ID NO: 82 - protein, 289 aa, Bacillus subtilis
  misc_feature (1)..(289): Bacillus subtilis NadC >gi|16079838|ref|NP_390664.1| nicotinate-nucleotide pyrophosphorylase [Bacillus subtilis subsp. subtilis str. 168]
MNHLQLKKLLNHFFLEDIGTGDLTSQSIFGEQSCEAEIVAKSEGIFAGAAIIKEGFSLLDENVQSILHKKDGDMLHKGEVIAELHGPAAALLSGERVVLNLIQRLSGIATMTREAVRCLDDEQIKICDTRKTTPGLRMLEKYAVRAGGGYNHRFGLYDGIMIKDNHIAACGSILEACKKARQAAGHMVNIEVEIETEEQLREAIAAGADVIMFDNCPPDTVRHFAKLTPANIKTEASGGITLESLPAFKGTGVNYISLGFLTHSVKSLDISMDVTLSNESVEECCYVNS

SEQ ID NO: 83 - protein, 279 aa, Corynebacterium glutamicum
  misc_feature (1)..(279): Corynebacterium glutamicum NadC >gi|41325293|emb|CAF19773.1| PUTATIVE NICOTINATE-NUCLEOTIDE PYROPHOSPHORYLASE [Corynebacterium glutamicum ATCC 13032]
MTTHIDRIVGAALSEDAPWGDITSDTFIPGSAQLSAKVVAREPGVFSGQALFDASFRLVDPRINASLKVADGDSFETGDILGTITGSARSILRSERIALNFIQRTSGIATLTSCYVAEVKGTKARIVDTRKTTPGLRIIERQAVRDGGGFNHRATLSDAVMVKDNHLAAIASQGLSITEALSNMKAKLPHTTHVEVEVDHIEQIEPVLAAGVDTIMLDNFTIDQLIEGVDLIGGRALVEASGGVNLNTAGKIASTGVDVISVGALTHSVHALDLGLDIF

SEQ ID NO: 84 - DNA, 20 bp, Artificial Sequence
  primer 10444
cggtaagtcccgtctagcct

SEQ ID NO: 85 - DNA, 25 bp, Artificial Sequence
  primer 10447
atgtttgcaaaacgattcaaaacct

SEQ ID NO: 86 - DNA, 37 bp, Artificial Sequence
  primer 11222
ttacaccgaatttctaataataaccgggcaggccatg

SEQ ID NO: 87 - DNA, 37 bp, Artificial Sequence
  primer 11223
ggcctgcccggttattattagaaattcggtgtaagag

SEQ ID NO: 88 - DNA, 40 bp, Artificial Sequence
  primer 11226
cttttacaccgaatttttaataataaccgggcaggccatg

SEQ ID NO: 89 - DNA, 37 bp, Artificial Sequence
  primer 11227
ggcctgcccggttattattaaaaattcggtgtaaaag

SEQ ID NO: 90 - DNA, 40 bp, Artificial Sequence
  primer 11230
cggatgaagcggaatgttaataataaccgggcaggccatg

SEQ ID NO: 91 - DNA, 37 bp, Artificial Sequence
  primer 11230
ggcctgcccggttattattaacattccgcttcatccg

SEQ ID NO: 92 - DNA, 38 bp, Artificial Sequence
  primer 11232
gaaaggtggtgaactactatgaaaacagcagcatacgc

SEQ ID NO: 93 - DNA, 40 bp, Artificial Sequence
  primer11233
gcgtatgctgctgttttcatagtagttcaccacctttctc

SEQ ID NO: 94 - DNA, 40 bp, Artificial Sequence
  primer 11234
cacttacaccgaacttctaataataaccgggcaggccatg

SEQ ID NO: 95 - DNA, 37 bp, Artificial Sequence
  primer 11235
ggcctgcccggttattattagaagttcggtgtaagtg

SEQ ID NO: 96 - DNA, 33 bp, Artificial Sequence
  primer 11341
aagggaggtttcatatgaaaattgttaaagatt

SEQ ID NO: 97 - DNA, 37 bp, Artificial Sequence
  primer 11342
tttaacaattttcatatgaaacctcccttaattctcg

SEQ ID NO: 98 - DNA, 35 bp, Artificial Sequence
  primer 11352
gtgaactactatgaaaattgtaaaaaactttattg

SEQ ID NO: 99 - DNA, 35 bp, Artificial Sequence
  primer 11352
ttacaattttcatagtagttcaccacctttctcta

SEQ ID NO: 100 - DNA, 35 bp, Artificial Sequence
  primer 11353
aagggaggtttcatatgaaaatcgttaaagacttc

SEQ ID NO: 101 - DNA, 35 bp, Artificial Sequence
  primer 11354
taacgattttcatatgaaacctcccttaattctcg

SEQ ID NO: 102 - DNA, 36 bp, Artificial Sequence
  primer 11159
gctacttactctcgagttactttttccagaaatcat

SEQ ID NO: 103 - DNA, 33 bp, Artificial Sequence
  primer 11160
gctaacttagcatatgatgacattgcaacaaca